(19) United States
(12) Patent Application Publication
(10) Pub. No.: US 2021/0275068 A1
(43) Pub. Date: Sep. 9, 2021

(54) UNIVERSAL TRANSFER ADAPTERS AND METHODS OF USING THE SAME

(71) Applicant: Magnolia Medical Technologies, Inc., Seattle, WA (US)

(72) Inventors: Jay M. MIAZGA, Langley, WA (US); Julie A. SCHNUR, Bellevue, WA (US); Shan E. GAW, Seattle, WA (US); Dylan GUELIG, Seattle, WA (US)

(73) Assignee: Magnolia Medical Technologies, Inc., Seattle, WA (US)

(21) Appl. No.: 17/193,930

(22) Filed: Mar. 5, 2021

Related U.S. Application Data
(60) Provisional application No. 62/986,244, filed on Mar. 6, 2020.

(51) Int. Cl. A61B 5/15

(52) U.S. Cl. CPC A61B 5/150534; A61B 5/150099

(57) ABSTRACT

An apparatus includes a housing defining an inner volume, a distal coupler at least temporarily coupled to a distal end portion of the housing and configured to be placed in fluid communication with a bodily fluid source, a fluid communicator disposed in the inner volume, and a lock coupled to the housing. The lock is transitionable between a first configuration in which the lock couples the distal coupler to housing such that a portion of the fluid communicator extends through a seal of the distal coupler to place the distal coupler in fluid communication with a proximal end portion of the housing and a second configuration in which the lock allows for removal of the distal coupler. The lock is configured to be transitioned back to the first configuration after removing the distal coupler to limit access to the fluid communicator via the distal end portion of the housing.

### CROSS-REFERENCE TO RELATED APPLICATIONS

This application claims priority to and the benefit of U.S. Provisional Patent Application No. 62/986,244, filed Mar. 6, 2020, entitled, “Universal Transfer Adapters and Methods of Using the Same,” the disclosure of which is incorporated herein by reference in its entirety.

### BACKGROUND

Embodiments described herein relate generally to the procurement of bodily fluid samples, and more particularly to fluid transfer adapters configured to reduce sources of touch point contamination.

Healthcare practitioners routinely perform various types of microbial as well as other broad diagnostic tests on patients using parenterally obtained bodily fluids. As bacterial culture testing and/or other advanced diagnostic technologies evolve and improve, the speed, accuracy (both sensitivity and specificity), and value of information that can be provided to clinicians continues to improve. Examples of diagnostic technologies that may be reliant on high quality (non-contaminated and/or unadulterated) bodily fluid samples can include but are not limited to microbial detection (e.g., culture testing), molecular diagnostics (e.g., molecular polymerase chain reaction (PCR), genetic sequencing (e.g., deoxyribonucleic acid (DNA), ribonucleic acid (RNA), whole blood (“culture free”) specimen analysis and associated technologies or next-generation sequencing (NGS)), biomarker identification, magnetic resonance and other magnetic analytical platforms, automated microscopy, spatial clone isolation, flow cytometry, morphokinetic cellular analysis, and/or other common or advanced/evolving technologies used to characterize patient specimens and/or to detect, identify, type, categorize, and/or characterize specific organisms, antibiotic susceptibilities, and/or the like.

Some known testing and/or diagnostic technologies, however, can be prone to contamination, which can lead to results that are inaccurate, distorted, adulterated, falsely positive, falsely negative, and/or otherwise not representative of the actual condition (or in vivo condition) of the patient. One source of inaccurate results from such testing is the presence of biological matter, which can include cells external to the intended source for sample procurement and/or other external contaminants inadvertently included in the bodily fluid sample being analyzed. For example, despite antiseptic preparation of the skin of an insertion site, tissue fragments, hair follicles, sweat glands, and/or other skin adnexal structures and/or microbes residing thereon (“dermally residing microbes”) can be dislodged during venipuncture and transferred into and/or otherwise included in the specimen to be analyzed, thereby contaminating the sample and/or potentially distorting the results of one or more tests performed on the sample.

While some known devices and/or systems can reduce the likelihood of contamination by, for example, diverting and sequestering an initial volume of bodily fluid, which is more likely to contain contaminants, other potential sources of contamination may remain. For example, some sample procurement equipment, supplies, and/or systems can include multiple user and/or fluidic interfaces (e.g., patient to needle, needle to transfer adapter, transfer adapter to sample vessel, catheter hub to syringe, syringe to transfer adapter, needle/tubing to sample vessels, and/or any other fluidic interface or any combination(s) thereof) that can introduce additional points of potential contamination (e.g., “touch point contamination”). In addition, some sample procurement equipment such as, for example, transfer adapters and/or the like can be designed for use with specific supplies, sample containers, culture bottles, and/or the like, which can reduce standardization and can increase a likelihood of improper, inefficient, contamination-prone, and/or unsafe use.

Accordingly, a need exists for improved apparatus, systems, and/or methods for reducing contamination (e.g., touch point contamination) of bodily fluid samples and/or equipment used to procure bodily fluid samples.

### SUMMARY

Apparatus and methods for universal transfer adapters configured to reduce sources of contamination such as, for example, touch point contamination are described herein. In some embodiments, an apparatus includes a housing, a distal coupler, a fluid communicator, and a lock. The housing has a proximal end portion and a distal end portion and defines an inner volume. The distal coupler is at least temporarily coupled to the distal end portion of the housing and is configured to be placed in fluid communication with a bodily fluid source. The fluid communicator is disposed in the inner volume of the housing. The lock is coupled to the housing is transitionable between a first configuration in which the lock couples the distal coupler to housing such that a portion of the fluid communicator extends through a seal of the distal coupler to place the distal coupler in fluid communication with a proximal end portion of the housing and a second configuration in which the lock allows for removal of the distal coupler. The lock is configured to be transitioned from the second configuration back to the first configuration after removing the distal coupler to limit access to the fluid communicator via the distal end portion of the housing.

### BRIEF DESCRIPTION OF THE DRAWINGS

FIGS. 1A and 1B are schematic illustrations of a transfer adapter in a first configuration and a second configuration, respectively, according to an embodiment.

FIG. 2 is a perspective view of a transfer adapter coupled to a fluid collection device, according to an embodiment.

FIG. 3 is a front view of the transfer adapter and fluid collection device of FIG. 2.

FIG. 4 is an exploded front view of the transfer adapter and fluid collection device of FIG. 2.

FIG. 5 is a cross-sectional view of the transfer adapter and fluid collection device of FIG. 1, taken along the line 5-5, and shown in a first configuration.

FIG. 6 is a cross-sectional view of the transfer adapter and fluid collection device of FIG. 2, taken along the line 6-6, and shown in the first configuration.

FIGS. 7 and 8 are cross-sectional views of the transfer adapter illustrating various interior features thereof.

FIG. 9 is a front view of the transfer adapter coupled to the fluid collection device and having been transitioned from the first configuration to a second configuration.

FIG. 10 is a cross-sectional view of the transfer adapter and fluid collection device of FIG. 9 and shown with a coupler of the transfer adapter removed from a housing of the transfer adapter.

FIG. 11 is a perspective view of a transfer adapter according to an embodiment.

FIGS. 12 and 13 are cross-sectional views of the transfer adapter of FIG. 11, shown in a first state and a second state, respectively.

FIGS. 14-16 are illustrations of a portion of a transfer adapter having various configurations, each according to a different embodiment.

FIGS. 17-27 are illustrations of a portion of a transfer adapter having one or more features configured to protect a user against accidental and/or undesirable contact with a fluid communicator of the transfer adapter, each according to a different embodiment.

FIGS. 28-34 are illustrations of a portion of a transfer adapter having one or more features configured to provide and/or enhance a user interface of the transfer adapter, each according to a different embodiment.

FIG. 35 is a flowchart illustrating a method of using a transfer adapter according to an embodiment.

FIG. 36 is a cross-sectional view of a portion of a syringe that includes, for example, an integrated adapter, according to an embodiment.

### DETAILED DESCRIPTION

Apparatus and methods for universal transfer adapters configured to reduce sources of contamination such as, for example, touch point contamination(s) are described herein. Any of the embodiments and/or methods described herein can be configured to transfer bodily fluids while reducing a number of user and/or fluidic interfaces that otherwise may be potential sources of contamination. The embodiments and/or methods described herein may also simplify and/or standardize at least a portion of a sample or specimen procurement process, which can increase an efficiency and predictability associated with sample or specimen collection. Moreover, the embodiments and/or methods described herein may increase user safety by limiting and/or reducing a likelihood of inadvertent “needle sticks” (e.g., the undesirable puncturing of skin by a needle) and/or other undesirable contact with bodily fluid or non-sterile (e.g., used) portions of the device.

In some embodiments, an apparatus includes a housing, a distal coupler, a fluid communicator, and a lock. The housing has a proximal end portion and a distal end portion and defines an inner volume. The distal coupler is at least temporarily coupled to the distal end portion of the housing and is configured to be placed in fluid communication with a bodily fluid source. The fluid communicator is disposed in the inner volume of the housing. The lock is coupled to the housing is transitionable between a first configuration in which the lock couples the distal coupler to housing such that a portion of the fluid communicator extends through a seal of the distal coupler to place the distal coupler in fluid communication with a proximal end portion of the housing and a second configuration in which the lock allows for removal of the distal coupler. The lock is configured to be transitioned from the second configuration back to the first configuration after removing the distal coupler to limit access to the fluid communicator via the distal end portion of the housing.

In some embodiments, an apparatus includes a housing, a fluid communicator, a stage, and a bias member. The housing has a proximal end portion and a distal end portion and defines an inner volume. The proximal end portion has a proximal coupler. The fluid communicator is disposed in the inner volume of the housing and fluidically coupled to the proximal coupler. The stage is disposed in the housing and is movable between a first position and a second position. The bias member is disposed in the housing and is in contact with a proximal side of the stage. The bias member is configured to bias the stage in the first position such that the stage substantially prevents access to the fluid communicator via the distal end portion of the housing. The bias member allows the stage to be moved to the second position in response to a force exerted on a distal side of the stage such that a portion of the fluid communicator extends through the stage, thereby allowing access to the fluid communicator via the distal end portion of the housing.

In some embodiments, a transfer adapter includes a housing with a proximal end portion and a distal end portion. A proximal coupler is disposed along the proximal end portion of the housing. The transfer adapter further includes a fluid communicator disposed in an inner volume of the housing and fluidically coupled to the proximal coupler. In some implementations, a method of using the transfer adapter includes coupling a fluid collection device to the proximal coupler of the transfer adapter. A lock coupled to the distal end portion of the housing is transitioned from a locked configuration to an unlocked configuration. A stage disposed in the inner volume of the housing is moved from a first position in which the stage limits access to the fluid communicator via the distal end portion of the housing to a second position in which at least a portion of the fluid communicator extends through the stage. A flow of bodily fluid is allowed to flow into or out of the fluid collection device coupled to the proximal coupler via the fluid communicator when the stage is in the second position.

As used in this specification and/or any claims included herein, the singular forms “a,” “an,” and “the” include plural referents unless the context clearly dictates otherwise. Thus, for example, the term “a member” is intended to mean a single member or a combination of members, “a material” is intended to mean one or more materials, and/or the like.

As used herein, “bodily fluid” can include any fluid obtained directly or indirectly from a body of a patient. For example, “bodily fluid” includes, but is not limited to, blood, cerebrospinal fluid, urine, bile, lymph, saliva, synovial fluid, serous fluid, pleural fluid, amniotic fluid, mucus, sputum, vitreous, air, and/or the like, or any combination thereof.

As used herein, the words “proximal” and “distal” refer to the direction closer to and away from, respectively, a user who would place a device into contact with a patient. Thus, for example, the end of a device first touching the body of a patient would be a distal end of the device, while the opposite end of the device (e.g., the end of the device being manipulated by the user) would be a proximal end of the device.

As used herein, the terms “about,” “approximately,” and/or “substantially” when used in connection with stated value(s) and/or geometric structure(s) or relationship(s) is intended to convey that the value or characteristic so defined is nominally the value stated or characteristic described. In some instances, the terms “about,” “approximately,” and/or “substantially” can generally mean and/or can generally contemplate a value or characteristic stated within a desirable tolerance (e.g., plus or minus 10% of the value or characteristic stated). For example, a value of about 0.01 can include 0.009 and 0.011, a value of about 0.5 can include 0.45 and 0.55, a value of about 10 can include 9 to 11, and a value of about 1000 can include 900 to 1100. Similarly, a first surface may be described as being substantially parallel to a second surface when the surfaces are nominally parallel. While a value, structure, and/or relationship stated may be desirable, it should be understood that some variance may occur as a result of, for example, manufacturing tolerances or other practical considerations (such as, for example, the pressure or force applied through a portion of a device, conduit, lumen, etc.). Accordingly, the terms “about,” “approximately,” and/or “substantially” can be used herein to account for such tolerances and/or considerations.

The embodiments described herein can be configured to transfer bodily fluid substantially free of contaminants to one or more fluid collection device(s). A “fluid collection device,” as used herein, can include, but is not limited to, any suitable vessel, container, reservoir, bottle, adapter, dish, vial, syringe, device, needle, lumen-defining device (e.g., sterile flexible tubing), diagnostic and/or testing machine, and/or the like. In some embodiments, a fluid collection device can be substantially similar to or the same as known sample containers such as, for example, a Vacutainer® (manufactured by Becton Dickinson and Company (BD)), a BacT/ALERT® SN or BacT/ALERT® FA (manufactured by Biomerieux, Inc.), and/or any suitable reservoir, vial, microvial, microliter vial, nanoliter vial, container, microcontainer, nanocontainer, and/or the like.

In some embodiments, a fluid collection device such as, for example, a sample reservoir, container, bottle, etc. can be devoid of contents prior to receiving a sample volume of bodily fluid. For example, in some embodiments, a fluid collection device or reservoir can define and/or can be configured to define or produce a vacuum or suction such as, for example, a vacuum-based collection tube (e.g., a Vacutainer®), a syringe, and/or the like. In other embodiments, a fluid collection device can include any suitable additives, culture media, substances, enzymes, oils, fluids, and/or the like. For example, a fluid collection device can be a sample or culture bottle including, for example, an aerobic or anaerobic culture medium. The sample or culture bottle can receive a bodily fluid sample, which can then be tested (e.g., after incubation and via in vitro diagnostic (IVD) tests, and/or any other suitable test) for the presence of, for example, Gram-Positive bacteria, Gram-Negative bacteria, yeast, fungi, and/or any other organism. If such a test of the culture medium yields a positive result, the culture medium can be subsequently tested using a PCR-based system to identify a specific organism. In some embodiments, a sample reservoir can include, for example, any suitable additive or the like in addition to or instead of a culture medium. Such additives can include, for example, heparin, citrate, ethylenediaminetetraacetic acid (EDTA), oxalate, sodium polyanethol sulfonate (SPS), and/or the like. In some embodiments, a fluid collection device can include any suitable additive or culture media and can be evacuated and/or otherwise devoid of air.

In general, the term “culture medium” can be used to describe a substance configured to react with organisms in a bodily fluid (e.g., microorganisms such as bacteria), while the term “additive” can be used to describe a substance configured to react with portions of the bodily fluid (e.g., constituent cells of blood, serum, synovial fluid, etc.). It should be understood, however, that a sample reservoir can include any suitable substance, liquid, solid, powder, lyophilized compound, gas, etc. Moreover, when referring to an “additive” within a sample reservoir, it should be understood that the additive could be or could include a culture medium, such as an aerobic culture medium and/or an anaerobic culture medium contained in a culture bottle, an additive, and/or any other suitable substance or combination of substances contained in a culture bottle and/or any other suitable reservoir such as those described above. That is to say, the embodiments described herein can be used with any suitable fluid reservoir or the like containing any suitable substance or combination of substances.

The embodiments described herein and/or portions thereof can be formed or constructed of one or more biocompatible materials. In some embodiments, the biocompatible materials can be selected based on one or more properties of the constituent material such as, for example, stiffness, toughness, durometer, bioreactivity, etc. Examples of suitable biocompatible materials include metals, glasses, ceramics, elastomers, thermoplastics, polymers, and/or the like. Examples of suitable metals include pharmaceutical grade stainless steel, gold, titanium, nickel, iron, platinum, tin, chromium, copper, and/or alloys thereof. A polymer material may be biodegradable or non-biodegradable. Examples of suitable biodegradable polymers include polylactides, polyglycolides, polylactide-co-glycolides (PLGA), polyanhydrides, polyorthoesters, polyetheresters, polycaprolactones, polyesteramides, poly(butyric acid), poly(valeric acid), polyurethanes, and/or blends and copolymers thereof. Examples of non-biodegradable polymers include nylons, polyesters, polycarbonates, polyacrylates, polysiloxanes (silicones), polymers of ethylene-vinyl acetates and other acyl substituted cellulose acetates, non-degradable polyurethanes, polystyrenes, polyvinyl chloride, polyvinyl fluoride, poly(vinyl imidazole), chlorosulphonate polyolefins, polyethylene, polyethylene oxide, polytetrafluoroethylene (PTFE), polyetheretherketone (PEEK), and/or blends and copolymers thereof.

The embodiments described herein and/or portions thereof can include components formed of one or more parts, features, structures, etc. When referring to such components it should be understood that the components can be formed by a singular part having any number of sections, regions, portions, and/or characteristics, or can be formed by multiple parts or features. For example, when referring to a structure such as a wall or chamber, the structure can be considered as a single structure with multiple portions, or as multiple, distinct substructures or the like coupled to form the structure. Thus, a monolithically constructed structure can include, for example, a set of substructures. Such a set of substructures may include multiple portions that are either continuous or discontinuous from each other. A set of substructures can also be fabricated from multiple items or components that are produced separately and are later joined together (e.g., via a weld, an adhesive, or any suitable method).

The embodiments herein, and/or the various features or advantageous details thereof, are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended to facilitate an understanding of ways in which the embodiments herein may be practiced and to further enable those of skill in the art to practice the embodiments herein. While some of the embodiments are described herein as being used for procuring bodily fluid for one or more culture sample testing, it should be understood that the embodiments are not limited to such a use. Any of the embodiments and/or methods described herein can be used to transfer a flow of bodily fluid to any suitable device that is placed in fluid communication therewith. Thus, while specific examples are described herein, the devices, methods, and/or concepts are not intended to be limited to such specific examples.

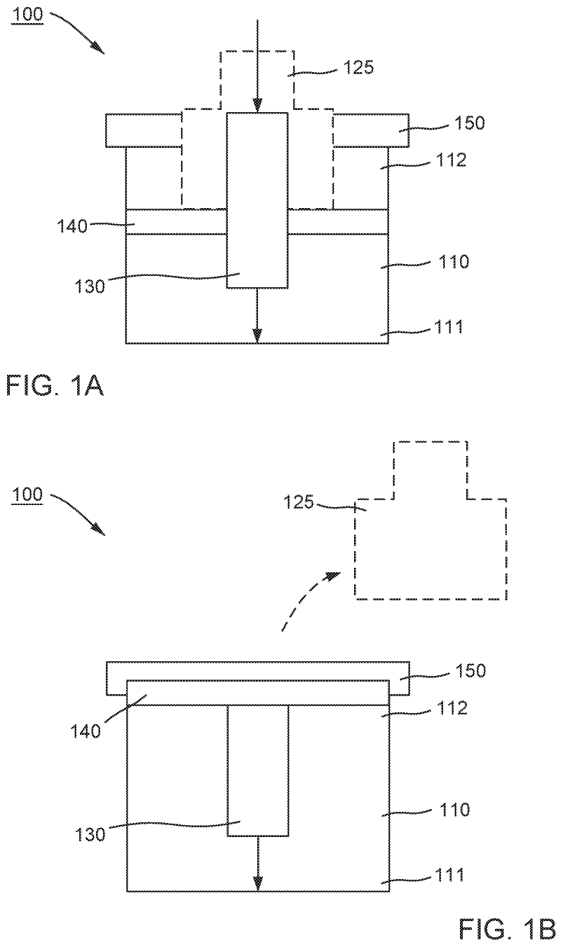

Referring to the drawings, FIGS. 1A and 1B illustrate a transfer device 100 according to an embodiment. The transfer device 100 (also referred to herein as “transfer adapter,” “adapter,” and/or “device”) can be any suitable shape, size, and/or configuration. In some implementations, the transfer adapter 100 is configured to transfer bodily fluids while reducing a number of user and/or fluidic interfaces that otherwise may be potential sources of contamination. More particularly, in some implementations, the transfer adapter 100 can be coupled to a fluid collection device (or any other suitable device) and used to transfer bodily fluid from a source (e.g., a bodily fluid source such as a vein of a patient) to the fluid collection device. In addition, the transfer adapter 100 can be used to transfer at least a portion of the bodily fluid from the fluid collection device to a second collection device or container (e.g., a sample bottle, culture bottle, and/or the like).

As shown, the transfer adapter 100 includes a housing 110, a fluid communicator 130, and a lock 150, and a stage 140. The housing 110 can be any suitable shape, size, and/or configuration. In some embodiments, the housing 110 can have a size and/or shape that is based at least in part on a size and/or shape of one or more devices configured to be used in conjunction with the transfer adapter 100, as described in further detail herein. The housing 110 includes a proximal end portion 111 and a distal end portion 112 and defines an inner volume. The proximal end portion 111 of the housing 110 is substantially open and is sized and configured to receive and/or configured to be physically and/or fluidically coupled, directly or indirectly, to one or more devices such as, for example, a fixed or removable coupler, a fluid collection device, a fluid transfer device, a needle, and/or the like. For example, the proximal end portion 111 of the housing 110 can be coupled to and/or can include a proximal coupler, which in turn, can be at least temporarily coupled to a fluid collection device. For example, the proximal coupler can be physically and fluidically coupled to a connector or coupler of a syringe via a threaded coupling, a luer-style coupling, and/or any other suitable connection. In other embodiments, the proximal coupler can be fixedly coupled or connected to the connector of the syringe (e.g., integrally or monolithically formed, pre-assembled, and/or the like) and/or to any other suitable device.

The proximal end portion 111 of the housing 110 (or a proximal coupler thereof) can be in fluid communication with the fluid communicator 130 disposed in the inner volume of the housing 110. As such, when the proximal end portion 111 of the housing 110 (or a proximal coupler thereof) is coupled to a syringe, manipulation of the syringe can result in a negative pressure differential and/or suction force operable to draw a fluid (e.g., bodily fluid) through the transfer adapter 100 (e.g., via the fluid communicator 130) and into the syringe or can result in a positive pressure differential and/or force operable to expel a fluid (e.g., bodily fluid) out of the syringe and through the transfer adapter 100 (e.g., via the fluid communicator 130).

In some implementations, the proximal end portion 111 of the housing 110 (or proximal coupler thereof) can be coupled, directly or indirectly, to a bodily fluid source. For example, in some implementations, the proximal end portion 111 of the housing 110 can include a proximal coupler such as, for example, a luer lock or the like that can be coupled to a corresponding coupler of a needle, lumen-containing device, and/or the like or combinations thereof In such implementations, the proximal coupler can receive a flow of bodily fluid from the bodily fluid source, which in turn, can be transferred through the transfer adapter 100 via the fluid communicator 130. In some implementations, the proximal coupler (or proximal end portion 111 of the housing 110) can be coupled, directly or indirectly, to a transfer, diversion, and/or sequestration device such as, for example, any of those described in U.S. Pat. No. 8,197,420 entitled, “Systems and Methods for Parenterally Procuring Bodily-Fluid Samples with Reduced Contamination,” filed Dec. 13, 2007 (“the '420 Patent”); U.S. Pat. No. 8,535,241 entitled, “Fluid Diversion Mechanism for Bodily-Fluid Sampling,” filed Oct. 22, 2012 (“the '241 Patent”); U.S. Pat. No. 9,022,950 entitled, “Fluid Diversion Mechanism for Bodily-Fluid Sampling,” filed Sep. 23, 2014 (“the '950 Patent”); U.S. Pat. No. 9,788,774 entitled, “Methods and Apparatus for Selectively Occluding the Lumen of a Needle,” filed Sep. 18, 2014 (“the '774 Patent”); U.S. Pat. No. 9,149,576 entitled, “Systems and Methods for Delivering a Fluid to a Patent with Reduced Contamination,” filed Oct. 9, 2013 (“the '576 Patent”); U.S. Pat. No. 9,204,864 entitled, “Fluid Diversion Mechanism for Bodily-Fluid Sampling,” filed Jul. 29, 2013 (“the '864 Patent”); U.S. Patent Publication No. 2018/0140240 entitled, “Systems and Methods for Sample Collection with Reduced Hemolysis,” filed Nov. 20, 2017 (“the '240 Publication”); U.S. Patent Publication No. 2018/0353117 entitled, “Fluid Control Devices and Methods of Using the Same,” filed Jun. 11, 2018 (“the '117 Publication”); U.S. Patent Publication No. 2019/0076074 entitled, “Fluid Control Devices and Methods of Using the Same,” filed Sep. 12, 2018 (“the '074 Publication”); U.S. Patent Publication No. 2019/0175087 entitled, “Fluid Control Devices and Methods of Using the Same,” filed Dec. 7, 2018 (“the '087 Publication”); U.S. Patent Publication No. 2019/0365303 entitled, “Fluid Control Devices and Methods of Using the Same,” filed May 30, 2019 (“the '303 Publication”); U.S. Patent Publication No. 2020/0289039 entitled, “Fluid Control Devices and Methods of Using the Same,” filed Mar. 11, 2020 (“the '039 Publication”); and/or U.S. patent application Ser. No. 17/119,732 entitled, “Fluid Transfer Devices with Integrated Flow-Based Assay and Methods of Using the Same,” filed Dec. 11, 2020 (“the '732 Application”), the disclosures of which are incorporated herein by reference in their entireties.

The distal end portion 112 of the housing 110 is substantially open and is sized and configured to receive and/or removably couple, directly or indirectly, to one or more devices such as, for example, a fixed or removable coupler, a fluid collection device, a fluid transfer device, a sample reservoir, a needle, and/or the like. In some embodiments, for example, the transfer adapter 100 can optionally include a distal coupler 125 that can be removably coupled to the distal end portion 112 of the housing 110. The optional distal coupler 125, in turn, can at least temporarily couple (directly or indirectly) to a bodily fluid source. For example, the optional distal coupler 125 can be a luer connector, a non-luer connector, and/or any other suitable coupling device that can be removably coupled to a lumen-containing device that is in fluid communication with a vein of a patient (e.g., a butterfly needle or other suitable type of needle, intravenous (IV) catheter, midline catheter, peripherally inserted central catheter (PICC), intermediary lumen-containing device, sterile flexible tubing, and/or the like). In other instances, a bodily fluid source need not be a patient and can, instead, be any suitable volume, reservoir, container, vial, dish, etc. that contains a bodily fluid. In some embodiments, the optional distal coupler 125 can be indirectly coupled to a bodily fluid source via one or more intermediate devices such as, for example, sterile tubing, a transfer device, a diversion device, a sequestration device, and/or one or more other intermediate devices. For example, the optional distal coupler 125 can be coupled to a transfer, diversion, and/or sequestration device such as any of those described in the '420 Patent, the '241 Patent, the '950 Patent, the '774 Patent, the '576 Patent, the '864 Patent, the '240 Publication, the '117 Publication, the '074 Publication, the '087 Publication, the '303 Publication, the '039 Publication, and/or the '732 Application.

As described in further detail herein, bodily fluid can be transferred from the patient and/or other bodily fluid source to the transfer adapter 100 via the optional distal coupler 125. In some implementations, the distal coupler 125 can be removed from the distal end portion 112 of the housing 110 after a desired amount of bodily fluid is transferred to the transfer adapter 100 or to a fluid collection device fluidically coupled to the transfer adapter 100 (e.g., a syringe). In some implementations, a second fluid collection device such as a sample bottle, culture bottle, evacuated container, and/or the like can be at least partially inserted into the distal end portion 112 of the housing 110 after the distal coupler 125 has been removed to allow a transfer of at least some of the collected bodily fluid (e.g., at least a portion of the bodily fluid contained in the fluid collection device coupled to the proximal end portion 111 of the housing 110 (or proximal coupler thereof)) through the transfer adapter 100 (e.g., via the fluid communicator 130) and into the second fluid collection device (e.g., sample bottle), as described in further detail herein.

The fluid communicator 130 is disposed within the inner volume of the housing 110. The fluid communicator 130 can be any suitable device configured to establish fluid communication between two or more components. For example, the fluid communicator 130 can be a conduit, a tube, and/or a lumen-defining device. In some embodiments, the fluid communicator 130 is a needle having a sharpened or beveled distal end or tip. In other embodiments, the fluid communicator 130 can be a needle or tube with a blunt distal end or tip. A proximal end portion of the fluid communicator is in fluid communication with the proximal end portion 111 of the housing (or a proximal coupler thereof). As described above, the proximal end portion 111 or the proximal coupler, in turn, can be coupled, directly or indirectly, to a fluid collection device such as a syringe, sample reservoir, needle, and/or the like. Thus, a lumen defined by the fluid communicator 130 can be placed in fluid communication with an inner volume or lumen of the fluid collection device allowing bodily fluid to be transferred therebetween. As an example, a proximal coupler of the housing 110 can be coupled to a syringe that can be manipulated to draw bodily fluid into the syringe via the fluid communicator 130 to expel bodily fluid from the syringe via the fluid communicator 130, as described in further detail herein with reference to specific embodiments.

In implementations including the optional distal coupler 125, the arrangement of the distal coupler 125 and the fluid communicator 130 can be such that at least a distal end portion of the fluid communicator 130 extends into and/or otherwise engages a portion of the distal coupler 125 when the distal coupler 125 is coupled to the distal end portion 112 of the housing 110 (see e.g., FIG. 1A). For example, a portion of the distal coupler 125 can be disposed in the inner volume of the housing 110 when coupled to the distal end portion 112 thereof such that the distal end portion of the fluid communicator 130 extends through and/or punctures a septum, seal, port, and/or the like of the distal coupler 125. Accordingly, when the distal coupler 125 is coupled to the housing 110, the lumen of the fluid communicator 130 is placed in fluid communication with the distal coupler 125 as well as, for example, a proximal coupler or other portion of the housing 110 to allow a flow of fluid (e.g., bodily fluid such as blood) to be transferred therebetween, as described in further detail herein.

Although not shown in FIGS. 1A and 1B, the transfer adapter 100 can include a sheath that is disposed in the inner volume and about or on at least a portion of the fluid communicator 130. In some embodiments, the sheath can be a relatively flexible cover or the like configured to surround at least a portion of the fluid communicator 130 to, for example, at least temporarily maintain a sterility of the fluid communicator 130 and/or to reduce a likelihood of undesirable user or patient contact with a portion of the fluid communicator 130. As described in further detail herein, the sheath can be configured to transition between a first state in which a distal end portion of the fluid communicator 130 extends through and/or is otherwise uncovered by the sheath and a second state in which the distal end portion of the fluid communicator 130 is disposed in and/or is otherwise covered by the sheath.

The lock 150 of the transfer adapter 100 can be any suitable shape, size, and/or configuration. In some embodiments, the lock 150 can be configured to selectively couple the optional distal coupler 125 to the distal end portion 112 of the housing 110. In some embodiments, the lock 150 can be transitioned between a first configuration, in which a portion of the lock 150 engages a portion of the optional distal coupler 125 thereby coupling the distal coupler 125 to the housing 110 (FIG. 1A), and a second configuration, in which the lock 150 does not engage the distal coupler 125 thereby allowing the distal coupler 125 to be removed from the distal end portion 112 of the housing 110 (FIG. 1B). For example, the lock 150 can include one or more shoulders configured to engage and/or contact one or more shoulders (or tabs) of the optional distal coupler 125 thereby coupling the distal coupler 125 to the housing 110 by maintaining a portion of the distal coupler 125 in the inner volume.

In some embodiments, the lock 150 can be transitioned between the first configuration and the second configuration by rotating the lock 150 (and/or a portion thereof) relative to the housing 110. The arrangement of the lock 150 can be such that rotating the lock 150 relative to the housing 110 rotates the one or more shoulders (or other portion(s)) of the lock 150 to a position that is misaligned relative to the one or more shoulders (or tabs or other portion(s)) of the optional distal coupler 125. In other words, rotating the lock 150 can be such that the lock 150 disengages and/or is removed from contact with the distal coupler 125, which in turn, allows the distal coupler 125 to be removed from the housing 110. While the lock 150 is described as being rotated relative to the housing 110 between the first configuration and the second configuration, it should be understood that a lock can be configured to transition in any suitable manner between any suitable number of configurations, states, and/or the like. For example, in some embodiments, a lock can be transitioned via rotational motion (e.g., as just described), translational motion (e.g., via a slider, a trigger, a button, and/or the like), and/or any other suitable change of state, configuration, arrangement, etc.

The transfer adapter 100 and/or the lock 150 or a lock assembly also includes the stage 140. The stage 140 can be a platform, disc, shelf, ring, plate, seal, etc. that is disposed in the inner volume of the housing 110 and movable between a first, distal, or biased position and a second, proximal, or unbiased position. Although not shown in FIGS. 1A and 1B, the stage 140 can include and/or can otherwise be in contact with a bias member or energy storage member on a proximal side or surface of the stage 140. In some embodiments, the bias member is a spring and/or any other energy storage member, bias member, etc. The bias member can be configured to place the stage 140 in a desired or biased position (e.g., the distal or first position). For example, the bias member can be configured to place the stage 140 in a desired, biased, or first position in which the stage 140 is at, near, and/or adjacent to the distal end portion 112 of the housing 110, as shown in FIG. 1B. Said another way, the bias member can bias the stage 140 in a distal position. Moreover, the stage 140 can be in a distal position relative to the fluid communicator 130 when in the biased or first configuration, state, and/or position, thereby limiting, blocking, and/or substantially preventing access to the fluid communicator 130 (FIG. 1B).

In some implementations, the stage 140 is disposed between the optional distal coupler 125 and, for example, a portion of the inner volume of the housing 110 and/or the fluid communicator 130 disposed in the portion of the inner volume of the housing 110 prior to the distal coupler 125 being coupled to the distal end portion 112 of the housing 110, as shown in FIG. 1B. As such, at least a portion of the distal coupler 125 (e.g., a portion or surface of the septum) can be placed in contact with the stage 140 when the distal coupler 125 is coupled to the housing 110 and pushes or moves the stage 140 toward the proximal end portion 111 of the housing 110 (e.g., moved from a first position to a second position, as shown in FIG. 1A). In other implementations, the stage 140 can be disposed between a fluid collection device such as a sample reservoir, culture bottle, evacuated container, etc. and the portion of the inner volume of the housing 110 and/or the fluid communicator 130 disposed in the portion of the inner volume of the housing 110. As such, at least a portion of the fluid collection device can be placed in contact with the stage 140 when coupled to and/or inserted into the distal end portion 112 of the housing 110 and pushes or moves the stage 140 from a distal or first position to a proximal or second position.

In some implementations, when coupled to the housing 110, the optional distal coupler 125, the fluid collection device, and/or any other suitable device can contact, push, and/or move the stage 140 in a proximal direction, which in turn, can transition the bias member to a second, unbiased, and/or compressed state or configuration. Moreover, when the stage 140 and bias member are in the second, unbiased, compressed and/or proximal position or state, at least a portion of the fluid communicator 130 can extend through and is distal to the stage 140, as shown in FIG. 1A. In some embodiments, this arrangement can allow the fluid communicator 130 to engage, puncture, and/or extend through a portion of the distal coupler 125 or fluid collection device (e.g., a septum, a frangible seal, a port, an inlet surface, etc.) thereby establishing fluid communication between the fluid communicator 130 and the fluid collection device and/or the optional distal coupler 125. Thus, the stage 140 can be, for example, a spring-loaded stage, platform, seal, and/or the like that can be biased in a position that limits and/or blocks access to the fluid communicator 130 in a first state or configuration and that allows access to the fluid communicator 130 in a second state or configuration.

In some implementations, the transfer adapter 100 can be pre-assembled, packaged, and/or shipped in a first state or configuration in which a fluid collection device is physically and/or fluidically coupled to the proximal end portion 111 of the housing 110. For example, the transfer adapter 100 can be pre-assembled, packaged, and/or shipped with a proximal coupler of the housing 110 coupled to a syringe or the like. In some implementations, the optional distal coupler 125 can also be coupled to the distal end portion 112 of the housing 110 with the lock 150 in the locked configuration.

An example of using the transfer adapter 100 with the optional distal coupler 125 and syringe pre-assembled is described below. It should be understood, however, that the process or method of use described below is presented by way of example only and not limitation. Other uses of the transfer adapter 100 are possible and may be described in further detail herein with reference to specific embodiments. For example, the transfer adapter 100 need not be pre-assembled but rather can be assembled and/or otherwise coupled to any desired device(s) by a user or healthcare professional.

When pre-assembled, the adapter 100 can be in a first configuration or state, as shown in FIG. 1A. For example, the lock 150 can be in the first configuration (e.g., a locked configuration) such that the optional distal coupler 125 is secured or coupled to the housing 110. As described above, the stage 140 is in a proximal, compressed, or second position when the distal coupler 125 is coupled to the housing 110 allowing at least a portion of the fluid communicator 130 to extend through and/or distal to the stage 140. In some implementations, a septum or other portion of the distal coupler 125 can engage a portion of the sheath at least partially surrounding the fluid communicator 130 to transfer the sheath to a compressed configuration, thereby exposing a portion of the fluid communicator 130. As such, the fluid communicator 130 extends outside of the sheath, distal to the stage 140, and punctures and/or extends through the septum. Thus, since the fluid communicator 130 is fluidically coupled to the proximal coupler, the fluid communicator 130 fluidically couples the distal coupler 125 and the proximal coupler.

In some implementations, a healthcare professional can remove the pre-assembled adapter 100, distal coupler 125, and syringe from a sterile packaging and can directly or indirectly fluidically couple the optional distal coupler 125 to a bodily fluid source. For example, the healthcare professional can couple the distal coupler 125 to a proximal port, coupler, and/or connector of a device, which in turn, is in fluid communication with a bodily fluid source such as a butterfly needle, intravenous catheter, and/or access device. In some instances, the distal coupler 125 can be coupled to an intermediate transfer, diversion, and/or sequestration device which can be configured to (i) receive a flow of bodily fluid from the bodily fluid source, (ii) divert an initial or first portion of the bodily fluid (that is more likely to include contaminants), (iii) sequester the initial or first portion of the bodily fluid, and (iv) allow a subsequent or second portion of the bodily fluid to flow through the device and to the optional distal coupler 125. While described as being coupled to the access device and/or intermediate transfer device, etc., in other implementations, the adapter 100 can be pre-assembled and/or packaged with any such device(s) connected to the distal coupler 125.

In the first configuration and/or state, the user or healthcare professional can manipulate the syringe by, for example, moving a plunger of the syringe in a proximal direction. The movement of the plunger, in turn, produces a negative pressure differential within the syringe that is operable to draw a volume of bodily fluid into the distal coupler 125, through the adapter 100 via the fluid communicator 130, and into an inner volume of the syringe.

After procuring a desired volume of bodily fluid in the syringe, the user or healthcare professional can manipulate the device 100 by transitioning the lock 150 from the first or locked configuration or state to the second or unlocked configuration or state. In some instances, the user can decouple and/or disconnect the distal coupler 125 from the bodily fluid source or device that is in fluid communication with the bodily fluid source prior to transitioning the lock 150. In other instances, the user need not decouple and/or disconnect the distal coupler 125. When the lock 150 is transitioned to the second or unlocked configuration or state, the user can decouple or remove the distal coupler 125 from the housing 110, thereby placing the transfer adapter 100 in a second configuration, as shown in FIG. 1B.

The arrangement of the distal coupler 125 can be such that removing the distal coupler 125 from the housing 110 withdraws the fluid communicator 130 from the distal coupler 125 and/or septum included therein. In some embodiments, the septum can be, for example, a self-healing septum, port, material, and/or the like that can transition or self-heal into a sealed state and/or configuration when the fluid communicator 130 is withdrawn, thereby preventing bodily fluid leaks associated with a portion of the fluid flow path distal to the septum.

The removal of the distal coupler 125 allows the stage 140 to move to its distal, biased, or first position. For example, a bias member (e.g., a spring) or the like can exert a force on the stage 140 to return it to the biased of first position. More specifically, the bias member is allowed to expand, which in turn, moves the stage 140 in a distal direction until the stage 140 and the bias member are in a biased or distal position. In some implementations, the stage 140 can be configured to selectively engage a portion of the sheath such that the distal movement of the stage 140 results in a distal movement of at least a portion of the sheath. As such, when the stage 140 is in the distal or first position, the sheath can cover at least a distal end portion of the fluid communicator 130. In some instances, after the distal coupler 125 is removed from the housing 110, the user can transition the lock 150 back to the first or locked configuration or state such that a portion of the lock 150 secures the stage 140 in the distal or biased position. As such, the stage 140 and the sheath can collectively limit and/or substantially prevent access to and/or contact with the fluid communicator 130.

In some implementations, it may be desirable to transfer at least a portion of the bodily fluid disposed in the syringe into a separate fluid collection device such as a sample bottle, culture bottle, testing apparatus, and/or the like. For example, in some instances, if not already in the second or unlocked configuration, the user can transition the lock 150 back to the second or unlocked configuration and can insert a portion of a culture bottle into the distal end portion 112 of the housing 110. In some embodiments, the size, shape, and/or configuration of at least the distal end portion 112 of the housing 110 is such that any suitable and/or commercially available culture bottle can be disposed in the housing 110. Moreover, a surface of the culture bottle can contact the stage 140 and can move and/or transition the stage 140 from the distal position toward the proximal position as the culture bottle is inserted into the housing 110. As such, an unsheathed portion of the fluid communicator 130 can extend distally relatively to the stage 140 and can puncture and/or otherwise be inserted into a portion of the culture bottle, thereby establishing fluid communication between the syringe and the culture bottle. Thus, the user can manipulate the plunger of the syringe or rely on a vacuum charge (e.g., negative pressure differential) of the culture bottle to transfer a desired volume of bodily fluid from the syringe to the culture bottle via the transfer adapter 100 without a need for additional devices and/or components that may otherwise introduce points of potential contamination.

While the use of the transfer adapter 100 with the optional distal coupler 125 is described above, in other implementations, the transfer adapter 100 can be used without the distal coupler 125. In such implementations, for example, the proximal end portion 111 of the housing 110 (or a proximal coupler thereof) can be coupled, directly or indirectly, to a bodily fluid source. For example, as described above, a proximal coupler can be coupled to an access device, a transfer device, and/or a combination thereof, which in turn, is/are in fluid communication with the bodily fluid source. In this implementation, the lock 150 can be in the unlocked configuration (or can be placed in the unlocked configuration) and a fluid collection device such as a sample bottle, culture bottle, testing apparatus, and/or the like can be inserted into the distal end portion 112 of the housing 110. As such, bodily fluid can flow from the bodily fluid source, through the transfer adapter 100, and into the culture bottle (or the like) in a manner substantially similar to that described with reference to the flow of bodily fluid from the syringe to the culture bottle when using the optional distal coupler 125.

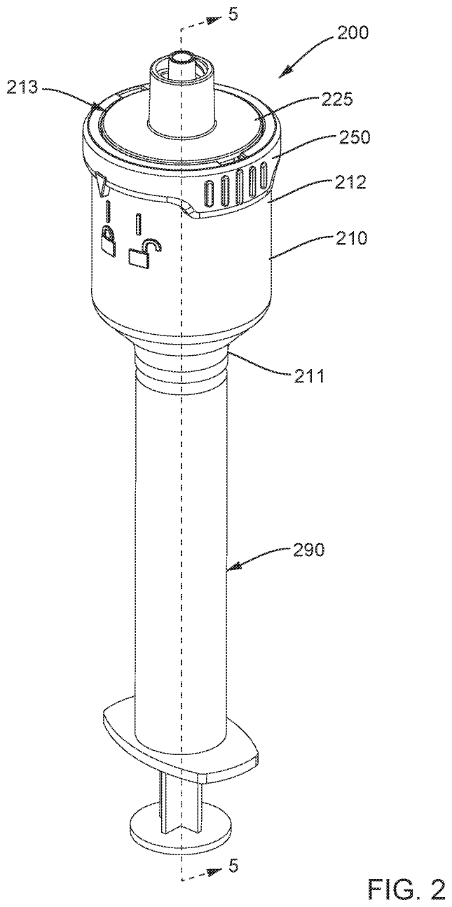

FIGS. 2-10 illustrate a transfer device 200 according to another embodiment. The transfer device 200 (also referred to herein as “transfer adapter,” “adapter,” and/or “device”) can be any suitable shape, size, and/or configuration. In some implementations, the transfer device 200 is configured to transfer bodily fluids while reducing a number of user and/or fluidic interfaces that otherwise may be potential sources of contamination. More particularly, in some implementations, the transfer device 200 can be coupled to a fluid collection device (or any other suitable device) and used to transfer bodily fluid from a source (e.g., a bodily fluid source such as a vein of a patient) to the fluid collection device. In addition, the transfer device 200 can be used to transfer at least a portion of the bodily fluid from the fluid collection device to a second collection device or container (e.g., a sample bottle, culture bottle, and/or the like).

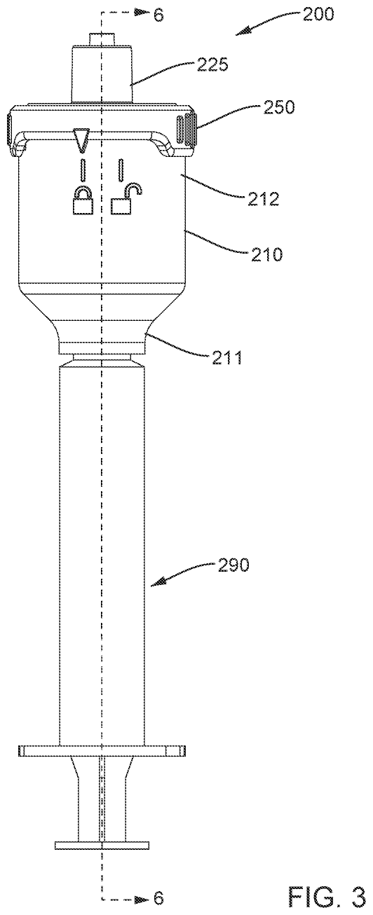

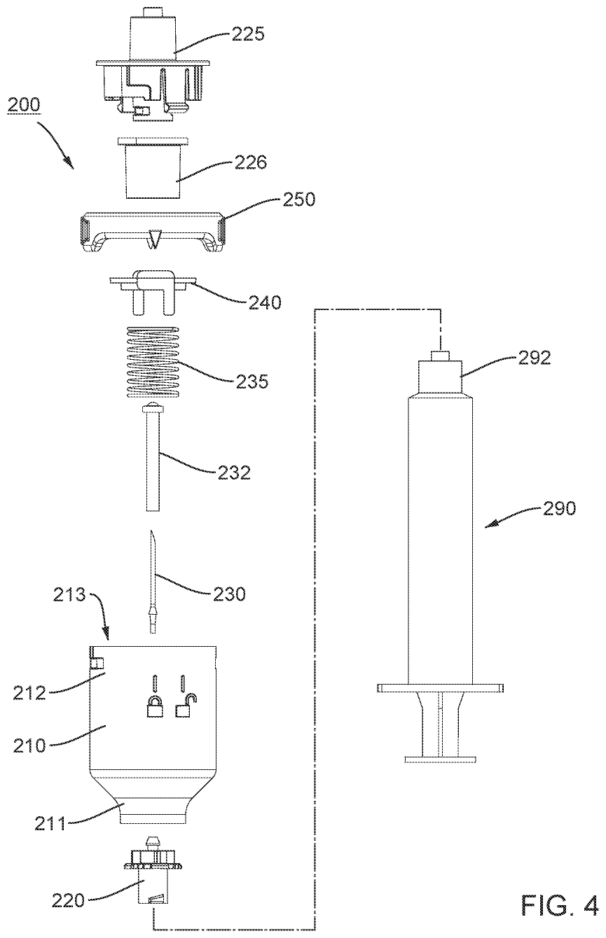

FIGS. 2 and 3 are a perspective view and a front view, respectively, of the transfer device 200 shown coupled to a syringe 290, as described in further detail herein. FIG. 4 is an exploded view of the transfer device 200. As shown, the transfer device 200 includes a housing 210, a proximal coupler 220, a distal coupler 225, a fluid communicator 230, and a lock 250.

The housing 210 can be any suitable shape, size, and/or configuration. In some embodiments, the housing 210 can have a size and/or shape that is based at least in part on a size and/or shape of one or more devices configured to be used in conjunction with the transfer device 200, as described in further detail herein.

The housing 210 includes a proximal end portion 211 and a distal end portion 212 and defines an inner volume. The proximal end portion 211 of the housing 210 is substantially open and is sized and configured to receive and/or configured to be coupled to the proximal coupler 220. The proximal coupler 220, in turn, is configured to at least temporarily couple to a fluid collection device. For example, in the embodiment shown in FIGS. 2-10, the proximal coupler 220 is configured to physically and fluidically couple to a connector 292 of a syringe 290 via a threaded coupling, a luer-style coupling, and/or any other suitable connection. In other embodiments, the proximal coupler 220 can be fixedly coupled or connected to the connector 292 of the syringe 290 (e.g., integrally or monolithically formed, pre-assembled, and/or the like).

The distal end portion 212 of the housing 210 is substantially open and is sized and configured to receive and/or removably couple to the distal coupler 225. The distal coupler 225, in turn, is at least temporarily coupled (directly or indirectly) to a bodily fluid source. For example, the distal coupler 225 can be a luer connector, a non-luer connector, and/or any other suitable coupling device that can be removably coupled to a lumen-containing device that is in fluid communication with a vein of a patient (e.g., a butterfly needle, intravenous (IV) catheter, peripherally inserted central catheter (PICC), intermediary lumen-containing device, and/or the like). In other instances, a bodily fluid source need not be a patient and can, instead, be any suitable volume, reservoir, container, vial, etc. that contains a bodily fluid. In some embodiments, the distal coupler 225 can be indirectly coupled to a bodily fluid source via one or more intermediate devices such as, for example, sterile tubing, a transfer, diversion, and/or sequestration device, and/or one or more other intermediate devices.

As described in further detail herein, bodily fluid can be transferred from the patient and/or other bodily fluid source to the transfer device 200 via the distal coupler 225. In some implementations, the distal coupler 225 is removed from the distal end portion 212 of the housing 210 after a desired amount of bodily fluid is transferred to the transfer device 200. In some implementations, a second fluid collection device such as a sample bottle, culture bottle, evacuated container, and/or the like can be at least partially inserted into the distal end portion 212 of the housing 210 after the distal coupler 225 has been removed to allow a transfer of at least some of the bodily fluid contained in the fluid collection device (e.g., the syringe 290) coupled to the proximal coupler 220 though the transfer device 200 and into the second fluid collection device (e.g., sample bottle), as described in further detail herein.

As shown in FIGS. 4-6, the fluid communicator 230 is disposed within the inner volume 213 of the housing 210. The fluid communicator 230 can be any suitable device configured to establish fluid communication between two or more components. For example, the fluid communicator 230 can be a conduit, a tube, and/or a lumen-defining device. More specifically, in the example shown in FIGS. 2-10, the fluid communicator 230 is a needle having, for example, a sharpened or beveled distal end or tip. In other embodiments, the fluid communicator 230 can have a blunt distal end or tip.

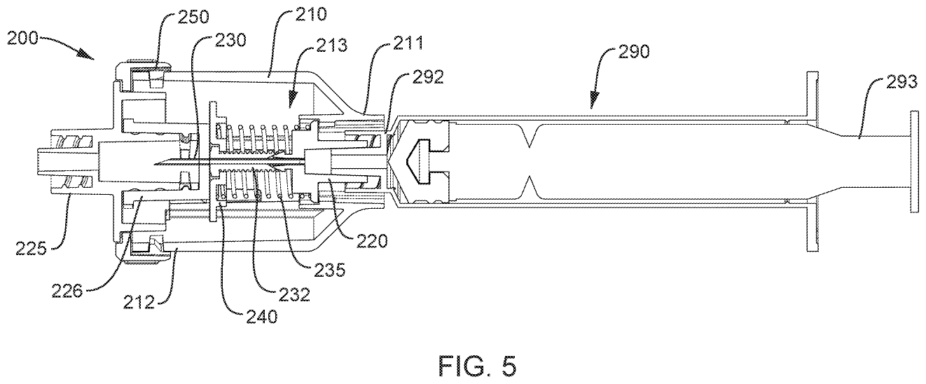

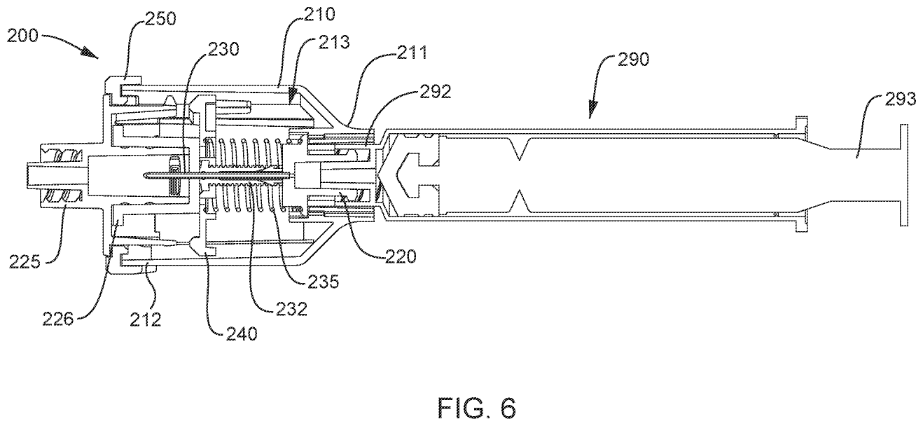

The fluid communicator 230 has a proximal end portion that is coupled to and in fluid communication with the proximal coupler 220 (see e.g., FIGS. 5 and 6). The proximal coupler 220, in turn, is in fluid communication with an inner volume of the syringe 290 when the proximal coupler 220 is coupled to the connector 292 of the syringe 290. Thus, a lumen defined by the fluid communicator 230 is fluidically coupled to an inner volume of the syringe 290 and the transfer device 200 and/or syringe 290 can be manipulated to transfer fluid therebetween, as described in further detail herein.

The fluid communicator 230 has a distal end portion that is configured to engage the distal coupler 225 when the distal coupler 225 is coupled to the distal end portion 212 of the housing 210. For example, as shown in FIGS. 5 and 6, a portion of the distal coupler 225 is disposed in the inner volume 213 when coupled to the distal end portion 212 of the housing 210 such that the distal end portion of the fluid communicator 230 extends through and/or punctures a septum 226 of the distal coupler 225. Accordingly, when the distal coupler 225 is coupled to the housing 210, the lumen of the fluid communicator 230 places the distal coupler 225 in fluid communication with the proximal coupler 220 to allow a flow of fluid (e.g., bodily fluid such as blood) to be transferred therebetween, as described in further detail herein.

The transfer device 200 also includes a sheath 232 that is disposed in the inner volume 213 and about or on at least a portion of the fluid communicator 230. In some embodiments, the sheath 232 can be a relatively flexible cover or the like configured to surround at least a portion of the fluid communicator 230 to, for example, at least temporarily maintain a sterility of the fluid communicator 230 and/or to reduce a likelihood of undesirable user or patient contact with a portion of the fluid communicator 230. As described in further detail herein, the sheath 232 can be configured to transition between a first state in which a distal end portion of the fluid communicator 230 extends through and/or is otherwise uncovered by the sheath 232 (see e.g., FIGS. 5 and 6) and a second state in which the distal end portion of the fluid communicator 230 is disposed in and/or is otherwise covered by the sheath 232 (see e.g., FIG. 10).

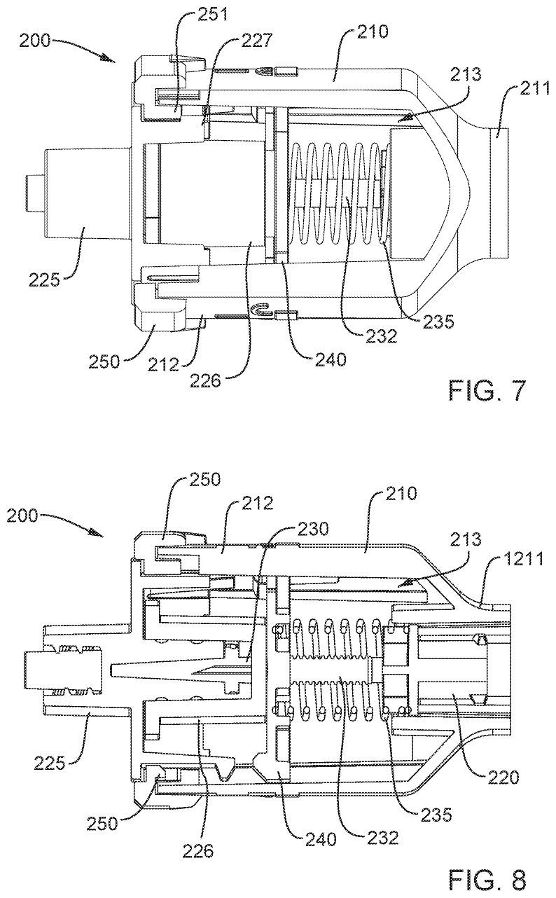

The lock 250 of the transfer device 200 can be any suitable shape, size, and/or configuration. In some embodiments, the lock 250 can be configured to selectively couple the distal coupler 225 to the distal end portion 212 of the housing 210. In some embodiments, the lock 250 can be transitioned between a first configuration, in which a portion of the lock 250 engages a portion of the distal coupler 225 thereby coupling the distal coupler 225 to the housing 210, and a second configuration, in which the lock 250 does not engage the distal coupler 225 thereby allowing the distal coupler 225 to be removed from the distal end portion 212 of the housing 210. For example, as shown in FIGS. 7 and 8, the lock 250 can include one or more shoulders 251 configured to engage and/or contact one or more shoulders 227 (or tabs) of the distal coupler 225 thereby coupling the distal coupler 225 to the housing 210 by maintaining a portion of the distal coupler 225 in the inner volume 213.

In some embodiments, the lock 250 can be transitioned between the first configuration and the second configuration by rotating the lock 250 (and/or a portion thereof) relative to the housing 210. The arrangement of the lock 250 can be such that rotating the lock 250 relative to the housing 210 rotates the one or more shoulders 251 of the lock 250 to a position that is misaligned relative to the one or more shoulders 227 (or tabs) of the distal coupler 225. In other words, rotating the lock 250 can be such that the lock 250 disengages and/or is removed from contact with the distal coupler 225, which in turn, allows the distal coupler 225 to be removed from the housing 210. While the lock 250 is described as being rotated relative to the housing 210 between the first configuration and the second configuration, it should be understood that a lock can be configured to transition in any suitable manner. For example, in some embodiments, a lock can be transitioned via rotational motion (e.g., as just described), translational motion (e.g., via a slider, a trigger, a button, and/or the like), and/or any other suitable change of state, configuration, arrangement, etc. While the lock 250 is shown and described as rotating, it should be understood that the lock 250 is not intended to be limited to such a configuration.

As shown in FIGS. 7 and 8, the transfer device 200 and/or the lock 250 or a lock assembly also includes a bias member 235 and a stage 240. In this embodiments, the stage 240 is a platform, disc, shelf, ring, plate, etc., that is disposed between the distal coupler 225 and the bias member 235 when the distal coupler 225 is coupled to the housing 210. More specifically, at least a portion of the distal coupler 225 (e.g., a portion or surface of the septum 226) is placed in contact with the stage 240 when the distal coupler 225 is coupled to the housing 210 and pushes or moves the stage 240 toward the proximal end portion 211 of the housing 210.

As shown, an opposite side of the stage 240 is in contact with the bias member 235. In some embodiments, the bias member 235 is a spring and/or any other energy storage member, bias member, etc. The bias member 235 is configured to place the stage 240 in a desired or biased position. For example, in this embodiment, the bias member 235 can be configured to place the stage 240 in a desired, biased, or first position in which the stage 240 is at, near, and/or adjacent to the distal end portion 212 of the housing 210 (see e.g., FIG. 10). Said another way, the bias member 235 can bias the stage 240 in a distal position. As shown in FIG. 10, the stage 240 can be in a distal position relative to the fluid communicator 230 when in the biased or first configuration, state, and/or position, thereby limiting, blocking, and/or substantially preventing access to the fluid communicator 230. Similarly stated, the stage 240 can include and/or can form a seal or the like that can isolate or substantially isolate the fluid communicator 230 in the inner volume of the housing 210. Said another way, the stage 240 can include and/or form a seal between the open distal end of the housing 210 and the fluid communicator 230, thereby limiting, blocking, and/or substantially preventing access to the fluid communicator 230 via the distal end of the housing 210 prior to inserting or coupling the distal coupler 225 to the housing 210.

As described in further detail herein, when coupled to the housing 210, the distal coupler 225 can contact, push, and/or move the stage 240 in a proximal direction, which in turn, can transition the bias member 235 to a second, unbiased, and/or compressed state or configuration, as shown in FIGS. 7 and 8. Moreover, when the stage 240 and bias member 235 are in the second, unbiased, compressed and/or proximal position or state, at least a portion of the fluid communicator 230 extends through and is distal to the stage 240, as shown in FIGS. 5 and 6. In some embodiments, this arrangement can allow the fluid communicator 230 to engage, puncture, and/or extend through a portion of the septum 226 of the distal coupler 225 thereby establishing fluid communication between the distal coupler 225 and the fluid communicator 230. Thus, the stage 240 can be, for example, a spring-loaded stage or platform that can be biased in a position that limits and/or blocks access to the fluid communicator 230 in a first state or configuration and that allows access to the fluid communicator 230 in a second state or configuration.

In some implementations, the transfer device 200 can be pre-assembled, packaged, and/or shipped in a first state or configuration in which the syringe 290 is coupled to the proximal coupler 220 and the distal coupler 225 is coupled to the distal end portion of the housing 210. In use, a healthcare professional can remove the pre-assembled device 200 and syringe 290 from a sterile packaging and can directly or indirectly fluidically couple the distal coupler 225 to a bodily fluid source. For example, in some instances, the healthcare professional can couple the distal coupler 225 to a proximal port, coupler, and/or connector of a device, which in turn, is in fluid communication with a bodily fluid source. In other instances, the device 200 can be pre-assembled and/or packaged with any suitable device connected to the distal coupler 225. As described above, the device can be, for example, a butterfly needle, intravenous catheter, and/or access device. In other instances, the device can be an intermediate transfer, diversion, and/or sequestration device which can be configured to receive a flow of bodily fluid, divert an initial portion of the bodily fluid (that is more likely to include contaminants), sequester the initial portion of the bodily fluid, and allow a subsequent portion of the bodily fluid to flow through the device and to the distal coupler 225.

In some embodiments, the transfer, diversion, and/or sequestration device can be any suitable device. For example, such a device can be similar to and/or substantially the same as any of the transfer, diversion, and/or sequestration devices described in the '420 Patent, the '241 Patent, the '950 Patent, the '774 Patent, the '576 Patent, the '864 Patent, the '240 Publication, the '117 Publication, the '074 Publication, the '087 Publication, the '303 Publication, the '039 Publication, and/or the '732 Application, incorporated by reference hereinabove.

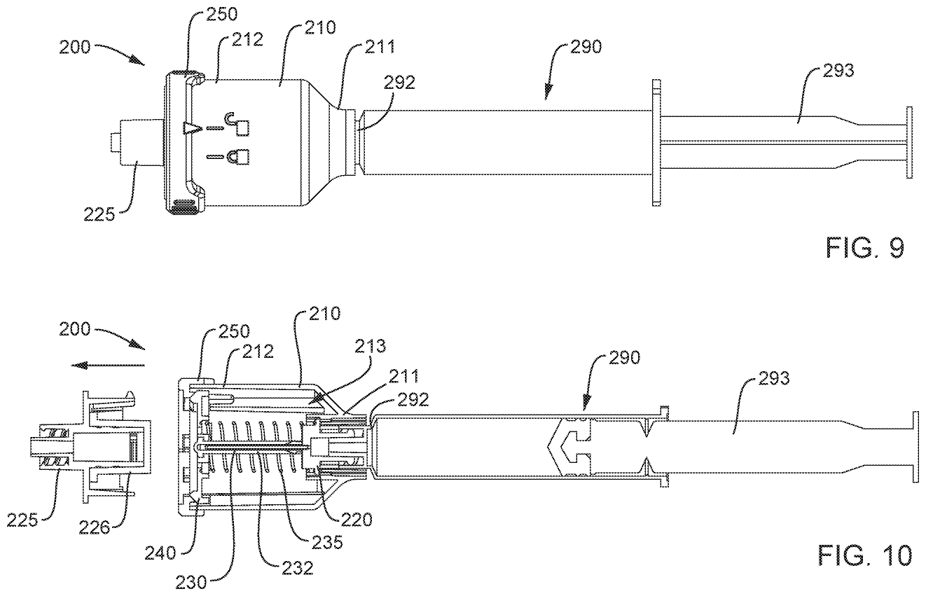

The lock 250 can be in the first configuration (e.g., a locked configuration) such that the distal coupler 225 is secured or coupled to the housing 210. As shown in FIGS. 5 and 6, the stage 240 is in a proximal or compressed position when the distal coupler 225 is coupled to the housing 210. Moreover, the septum 226 of the distal coupler 225 can engage a portion of the sheath 232 to transfer the sheath 232 to a compressed configuration. As such, the fluid communicator 230 extends outside of the sheath 232, distal to the stage 240, and punctures and/or extends through the septum 226. Since the fluid communicator 230 is fluidically coupled to the proximal coupler 220, the fluid communicator 230 establishes fluid communication between the distal coupler 225 and the proximal coupler 220, as shown in FIGS. 5 and 6. In this configuration and/or state, the user or healthcare professional can manipulate the syringe 290 by, for example, moving a plunger 293 of the syringe 290 in a proximal direction (see e.g., FIGS. 9 and 10). The movement of the plunger 293, in turn, produces a negative pressure differential within the syringe 290 that is operative in drawing a volume of bodily fluid into the distal coupler 225, through the fluid communicator 230, through the proximal coupler 220 and the connector 292, and into an inner volume of the syringe 290.

After procuring a desired volume of bodily fluid in the syringe 290, the user or healthcare professional can manipulate the device 200 by transitioning the lock 250 from the first or locked configuration or state to the second or unlocked configuration or state, as shown in FIG. 9. In some instances, the user can decouple and/or disconnect the distal coupler 225 from the bodily fluid source or device that is in fluid communication with the bodily fluid source prior to transitioning the lock 250. In other instances, the user need not decouple and/or disconnect the distal coupler 225.

When the lock 250 is transitioned to the second or unlocked configuration or state, the user can decouple or remove the distal coupler 225 from the housing 210, as indicated by the arrow in FIG. 10. The arrangement of the distal coupler 225 can be such that removing the distal coupler 225 from the housing 211 withdraws the fluid communicator 230 from the septum 226. In some embodiments, the septum 226 can be, for example, a self-healing septum, port, material, and/or the like that can transition or self-heal into a sealed state and/or configuration when the fluid communicator 230 is withdrawn, thereby preventing bodily fluid leaks associated with a portion of the fluid flow path distal to the septum 226.

The removal of the distal coupler 225, in turn, allows the bias member 235 (e.g., a spring) to return to a biased or initial configuration. More specifically, in this embodiment, the bias member 235 is allowed to expand, which in turn, moves the stage 240 in a distal direction until the stage 240 and the bias member 235 are in a biased or distal position. Moreover, the stage 240 can be configured to selectively engage a portion of the sheath 232 such that the distal movement of the stage 240 results in a distal movement of at least a portion of the sheath 232. As shown, when the stage 240 is in the distal position, the sheath 232 can completely cover at least a distal end portion of the fluid communicator 230. In some instances, after the distal coupler 225 is removed from the housing 210, the user can transition the lock 250 back to the first or locked configuration or state such that a portion of the lock 250 secures the stage 240 in the distal or biased position shown in FIG. 10. As such, the stage 240 and the sheath 232 can collectively limit and/or substantially prevent access to and/or contact with the fluid communicator 230.

In some implementations, it may be desirable to transfer at least a portion of the bodily fluid disposed in the syringe 290 into a separate fluid collection device such as a sample bottle, culture bottle, testing apparatus, and/or the like. For example, in some instances, if not already in the second or unlocked configuration, the user can transition the lock 250 back to the second or unlocked configuration and can insert a portion of a culture bottle into the distal end portion 212 of the housing 210. In some embodiments, the size, shape, and/or configuration of at least the distal end portion 212 of the housing 210 is such that any suitable and/or commercially available culture bottle can be disposed in the housing 210. Moreover, a surface of the culture bottle can contact the stage 240 and can move and/or transition the stage 240 from the distal position toward the proximal position as the culture bottle is inserted into the housing 210. As such, an unsheathed portion of the fluid communicator 230 can extend distally relatively to the stage 240 and can puncture and/or otherwise be inserted into a portion of the culture bottle, thereby placing the syringe 290 in fluid communication with the culture bottle. Thus, the user can manipulate the plunger 293 of the syringe 290 or rely on the vacuum charge (e.g., negative pressure differential) of the culture bottle to transfer a desired volume of bodily fluid from the syringe 290 to the culture bottle via the transfer device 200 without a need for additional devices and/or components that may otherwise introduce points of potential contamination.

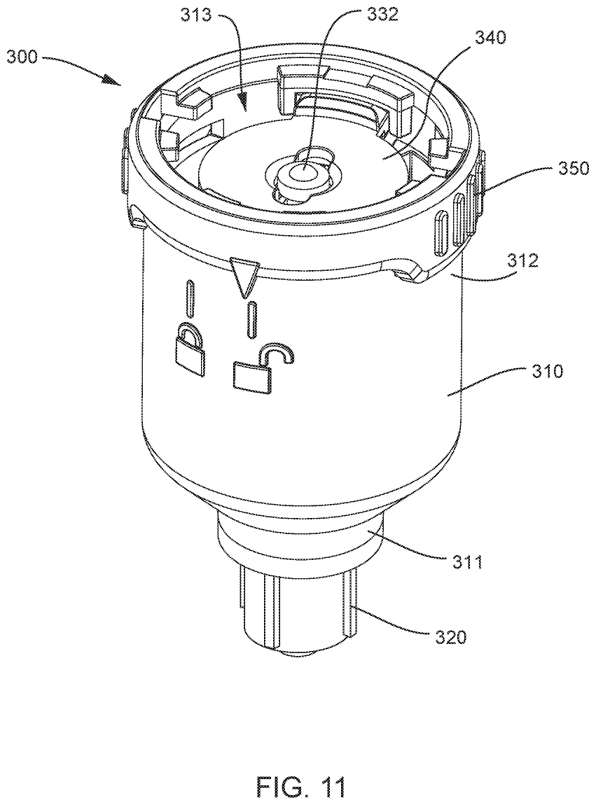

FIGS. 11-13 are illustrations of a transfer device 300 according to another embodiment. The transfer device 300 (also referred to herein as “transfer adapter,” “adapter,” and/or “device”) can be any suitable shape, size, and/or configuration. In some implementations, the transfer device 300 is configured to transfer bodily fluids while reducing a number of user and/or fluidic interfaces that otherwise may be potential sources of contamination. More particularly, in some implementations, the transfer device 300 can be coupled to a fluid collection device (or any other suitable device) and used to transfer bodily fluid from a source (e.g., a bodily fluid source such as a vein of a patient) to the fluid collection device. In addition, the transfer device 300 can be used to transfer at least a portion of the bodily fluid from the fluid collection device to a second collection device or container (e.g., a sample bottle, culture bottle, and/or the like). Portions and/or aspects of the transfer device 300 and/or portions thereof can be similar to or substantially the same as portions and/or aspects of the transfer devices 100 and/or 200 described above. Accordingly, such portions and/or aspects may not be described in further detail herein.

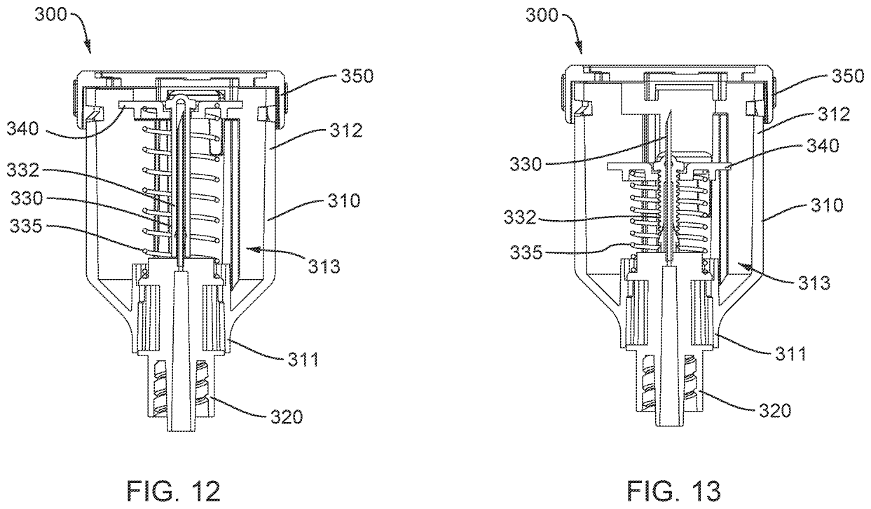

FIG. 11 is a perspective view of the transfer device 300. FIGS. 12 and 13 are cross-sectional views of the transfer device 300 in a first configuration and a second configuration, respectively. As shown, the transfer device 300 includes a housing 310, a proximal coupler 320, a fluid communicator 330, a sheath 332, a bias member 335, a stage 340, and a lock 350.

The housing 310 can be any suitable shape, size, and/or configuration. In some embodiments, the housing 310 can have a size and/or shape that is based at least in part on a size and/or shape of one or more devices configured to be used in conjunction with the transfer device 300. In some embodiments, the housing 310 is similar to or substantially the same as the housing 210 described above with reference to FIGS. 2-10. Accordingly, while portions of the housing 310 may be identified, such similar portions of the housing 310 are not described in further detail herein.

The housing 310 includes a proximal end portion 311 and a distal end portion 312 and defines an inner volume. The proximal end portion 311 of the housing 310 is substantially open and is sized and configured to receive and/or configured to be coupled to a proximal coupler 320. The proximal coupler 320, in turn, is at least temporarily coupled (directly or indirectly) to a bodily fluid source. For example, the proximal coupler 320 can be coupled and/or connected to a lumen-containing device that is in fluid communication with a vein of a patient (e.g., a butterfly needle, IV catheter, PICC line, intermediary lumen-containing device, and/or the like). In some embodiments, the proximal coupler 320 can be indirectly coupled to a bodily fluid source via one or more intermediate devices such as, for example, sterile tubing, a transfer, diversion, and/or sequestration device, and/or one or more other intermediate devices. For example, the proximal coupler 320 can be coupled to a fluid transfer device such as any of those described in the '420 Patent, the '241 Patent, the '950 Patent, the '774 Patent, the '576 Patent, the '864 Patent, the '240 Publication, the '117 Publication, the '074 Publication, the '087 Publication, the '303 Publication, the '039 Publication, and/or the '732 Application. In other embodiments, the proximal coupler 320 can be coupled to any suitable device. Thus, while the distal coupler 225 was described above as establishing fluid communication between the bodily fluid source and the transfer device 200, in this embodiments, the proximal coupler 320 establishes fluid communication between the bodily fluid source and the transfer device 300.

The distal end portion 312 of the housing 310 is substantially open and is sized and configured to receive a fluid collection device such as, for example, a sample bottle, a culture bottle, an evacuated container, and/or the like. While the device 200 was described above as including a distal coupler 225 that was removably coupled to the housing 210, in the embodiment shown in FIGS. 11-13, the transfer device 300 does not include and/or need not include a distal coupler. In this embodiment, for example, the transfer device 300 can be configured to transfer a flow of bodily fluid that is received by the proximal coupler 320, that flows through the fluid communicator 330, and that flows into a fluid collection device at least partially inserted into the distal end portion 312 of the housing 310, as described in further detail herein.

As shown in FIGS. 12 and 13, the fluid communicator 330 is disposed within the inner volume 313 of the housing 310. The fluid communicator 330 can be any suitable device configured to establish fluid communication between two or more components. For example, the fluid communicator 330 can be a conduit, a tube, a needle, a lumen-defining device, and/or the like. The fluid communicator 330 has a proximal end portion that is coupled to and in fluid communication with the proximal coupler 320, which in turn, is fluidically coupleable to a fluid transfer device such as those described above. The fluid communicator 330 has a distal end portion that is configured to engage a portion of a fluid collection device when the fluid collection device is at least partially inserted into the distal end portion 312 of the housing 310. Thus, a lumen defined by the fluid communicator 330 is configured to fluidically couple the proximal coupler 320 to a fluid collection device at least partially disposed in the housing 310. The transfer device 300 also includes a sheath 332 that is disposed in the inner volume 313 and about or on at least a portion of the fluid communicator 330 (see e.g., FIGS. 12 and 13). In some embodiments, the fluid communicator 330 and the sheath 332 can be similar in at least form and/or function to the fluid communicator 230 and the sheath 232, respectively, described above and thus, are not described in further detail herein.

The lock 350 of the transfer device 300 can be any suitable shape, size, and/or configuration. In some embodiments, the lock 350 can be similar in at least form and/or function to the lock 250 and thus, portions and/or aspects of the lock 350 are not described in further detail herein. The lock 350 is configured to be transitioned between a first configuration and a second configuration. While the lock 250 was described above as coupling the distal coupler 225 to the housing 210 when in the first or locked configuration, in the embodiment shown in FIGS. 11-13, the transfer device 300 does not include and/or need not be coupled to a distal coupler. Similar to the lock 250, however, the lock 350 is configured to lock the stage 340 in a desired position when in the first or locked configuration and is configured to release and/or allow the stage 340 to move when in the second or unlocked configuration, as described in further detail herein.

The stage 340 can be any suitable shape size, and/or configuration. For example, in some embodiments, the stage 340 can be a platform, disc, shelf, ring, plate, etc., that is configured to selectively limit access to the fluid communicator 330, as described above with reference to the stage 240. As shown in FIGS. 12 and 13, a proximal side or surface of the stage 340 is in contact with the bias member 335. In some embodiments, the bias member 335 is a spring and/or any other energy storage member, bias member, etc. The bias member 335 is configured to place the stage 340 in a desired or biased position. For example, the bias member 335 can be configured to place the stage 340 in a desired, biased, or first position in which the stage 340 is at, near, and/or adjacent to the distal end portion 312 of the housing 310 (see e.g., FIG. 12). As described above with reference to the stage 240, the stage 340 is in a distal position relative to the fluid communicator 330 when in the biased or first configuration, state, and/or position, thereby limiting, blocking, and/or substantially preventing access to the fluid communicator 330. Similarly stated, the stage 340 can include and/or can form a seal or the like that can isolate or substantially isolate the fluid communicator 330 in the inner volume of the housing 310. Said another way, the stage 340 can include and/or form a seal between the open distal end of the housing 310 and the fluid communicator 330, thereby limiting, blocking, and/or substantially preventing access to the fluid communicator 330 via the distal end of the housing 310.

As described in further detail herein, when a fluid collection device is at least partially inserted into the housing 310, a surface of the fluid collection device can contact, push, and/or move the stage 340 in a proximal direction, which in turn, can transition the bias member 335 to a second, unbiased, and/or compressed state or configuration, as shown in FIG. 13. When the stage 340 and bias member 335 are in the second, unbiased, compressed and/or proximal position or state, at least a portion of the fluid communicator 330 extends through and is distal to the stage 340, which allows the fluid communicator 330 to engage, puncture, and/or extend through a surface of the fluid collection device thereby establishing fluid communication between the proximal coupler 320 and the fluid collection device (not shown), as described in detail above with reference to the device 200.

In some implementations, the transfer device 300 can be packaged and/or shipped in a first state or configuration in which the stage 340 is in a distal position thereby limiting access to the fluid communicator 330. In some implementations, the transfer adapter 300 or device can be coupled to or pre-assembled with a fluid transfer device, diversion device, sequestration device, etc. connected to the proximal coupler 320. In other embodiments, the transfer adapter 300 or device is packaged independent from other devices such as a fluid transfer device.

In use, a healthcare professional can remove the device 300 from a sterile packaging and can directly or indirectly fluidically couple the proximal coupler 320 to a bodily fluid source. For example, in some instances, the healthcare professional can couple the proximal coupler 320 to a proximal port, coupler, and/or connector of a diversion and/or sequestration device, such as any of those described in the '420 Patent, the '241 Patent, the '950 Patent, the '774 Patent, the '576 Patent, the '864 Patent, the '240 Publication, the '117 Publication, the '074 Publication, the '087 Publication, the '303 Publication, the '039 Publication, and/or the '732 Application. The diversion and/or sequestration device (referred to as “diversion device”), in turn, is in fluid communication with a bodily fluid source (e.g., via a butterfly needle, IV catheter, PICC line, midline, access device, and/or the like).

In some implementations, the user or healthcare professional can manipulate the diversion device to initiate a flow of bodily fluid into the diversion device. The diversion device can be configured to automatically or manually (e.g., in response to user intervention) divert and sequester an initial portion of the bodily fluid transferred into the diversion device. Once the initial portion of the bodily fluid is sequestered, the diversion device can automatically or manually allow a subsequent flow of bodily fluid through the diversion device and into the proximal coupler 320. In some implementations, the proximal coupler 320 can be coupled to the diversion device prior to the diversion device receiving the flow of bodily fluid and a flow of bodily fluid can be drawn into and/or through the diversion device in response to a fluid collection device being at least partially inserted into the distal end portion 312 of the housing 310. In other implementations, the proximal coupler 320 of the transfer device 300 can be coupled to the diversion device after the diversion device has sequestered an initial portion of the bodily fluid.

As described above, the transfer device 300 can be in the first configuration and/or state prior to use. As such, the lock 350 is in the first configuration (e.g., a locked configuration) such that the stage 340 is in a distal position relative to the fluid communicator 330 thereby blocking and/or limiting access thereto, as shown in FIG. 12. After connecting the proximal coupler 320 to the diversion device, the user or healthcare professional can transition the transfer device 300 to a second configuration and/or state. For example, in some embodiments, the user can rotate and/or otherwise transition the lock 350 from the first or locked configuration to the second or unlocked configuration. As described above, when the lock 350 is in the second or unlocked configuration, the stage 340 is allowed to move relative to the fluid communicator 330 (e.g., proximally) to allow access thereto.

With the device 300 in the second configuration and/or state (e.g., when the lock 350 is in the unlocked configuration), the user can insert a portion of a fluid collection device into and/or through the distal end portion 312 of the housing 310. The fluid collection device can be, for example, any suitable and/or commercially available culture bottle, sample bottle, evacuated container, etc. As described above with reference to the device 200, inserting the fluid collection device into the housing 310 is such that a surface of the fluid collection device contacts a distal side or surface of the stage 340 and moves and/or transitions the stage 340 from the distal position (FIG. 12) toward the proximal position (FIG. 13) as the fluid collection device is advanced into the housing 310. As such, an unsheathed portion of the fluid communicator 330 can extend distally relatively to the stage 340 and can puncture and/or otherwise be inserted through a surface of the fluid collection device, thereby placing the proximal coupler 320 in fluid communication with the fluid collection device (e.g., via the fluid communicator 330).

With the fluid communicator 330 in fluid communication with the fluid collection device, the transfer device 300 can be configured to transfer bodily fluid from the diversion device coupled to the proximal coupler 320 and into the fluid collection device. As described above, in some implementations, the transfer device 300 can be coupled to the diversion device prior to or after to the diversion device receives a flow of bodily fluid. In some implementations, the fluid collection device can define a negative pressure and/or can otherwise be at least partially evacuated that results in a suction force being exerted through the fluid communicator 330 when the fluid communicator 330 punctures and/or is otherwise inserted into the fluid collection device. The suction force, in turn, can be operable to draw bodily fluid into the diversion device, which can automatically divert and sequester an initial volume of bodily fluid and once sequestered, can allow a subsequent flow of bodily fluid to bypass the sequestered initial volume and flow through the diversion device. Thus, the transfer device 300 can receive the subsequent flow of bodily fluid and can transfer the flow into the fluid collection device (e.g., via the proximal coupler 320 and the fluid communicator 330). In some instances, sequestering the initial volume of bodily fluid can also sequester contaminants that may be contained in the initial volume such that the subsequent flow of bodily fluid is substantially free of contamination. Moreover, limiting access to the fluid communicator 330 prior to inserting the fluid collection device into the housing 310 can also mitigate and/or eliminate a source of potential contamination. Thus, the bodily fluid transferred into the fluid collection device has a reduced likelihood of contamination and/or is substantially free of contamination.

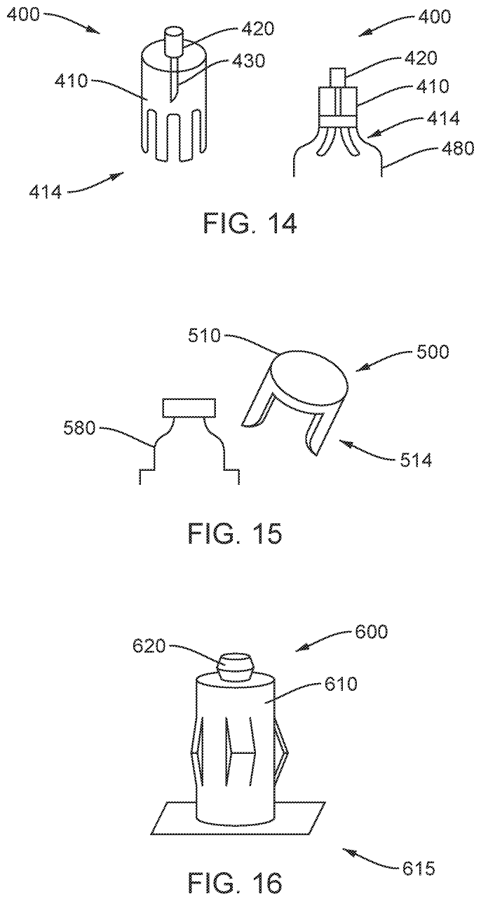

While the transfer devices 100, 200, and/or 300 have been particularly shown and described above, it should be understood that the transfer devices 100, 200, and/or 300 are presented by way of example only and not limitation. Various changes and/or modifications may be made to facilitate the use and/or compatibility of the devices and/or portions or aspects thereof. For example, FIGS. 14-34 illustrate portions and/or features of various transfer devices, one or more of which may be incorporated into the transfer devices 100, 200, and/or 300. Although not shown or described in detail herein, it should be understood that the transfer devices illustrated in FIGS. 14-34 can include any of the features, components, portions, etc. of the transfer devices 100, 200, and/or 300 and may be used in conjunction with any of the fluid collection devices, diversion devices, sequestration devices, etc., described herein above.

FIG. 14 illustrates a portion of a transfer device 400 according to an embodiment. The transfer device 400 includes a housing 410 that has a proximal coupler 420 and a fluid communicator 430 disposed within the housing 410 and in fluid communication with the proximal coupler 420. In this example, the transfer device 410 includes a set of flexible fingers, flanges, arms, extensions, etc. (referred to herein as “fingers 414”). As shown, the flexible fingers 414 can be configured to flex, bend, and/or elastically deform in response to a fluid collection device 480 being inserted into the housing 410. In some implementations, the flexible fingers 414 can allow fluid collection devices having various sizes and/or shapes to be inserted into the housing 410. In addition, in some implementations, the flexible fingers 414 can exert a friction force of a surface of the fluid collection device that can help secure the fluid collection device in the housing 410.

FIG. 15 illustrates a portion of a transfer device 500 according to another embodiment. The transfer device 500 includes a housing 510 that includes a set of flexible fingers 514, similar to the flexible fingers 414 described above. In this example, the flexible fingers 514 can include a smooth, rounded, and/or curved inner surface that can facilitate the insertion of a fluid collection device 580 into the transfer device 500. In some implementations, the inner surface of the fingers 514 can include a surface finish or texture configured to increase an amount of friction between the inner surface and an outer surface of the fluid collection device 580. In some implementations, the fingers 514 can be relatively rigid and the inner surface of the fingers can be formed with a relatively soft or pliable material that can at least partially conform to the outer surface of the fluid collection device 580 when inserted therein.

FIG. 16 illustrates a portion of a transfer device 600 according to another embodiment. The transfer device 600 includes a housing 610 that has a proximal coupler 620 and a fluid communicator (not shown) that is disposed within the housing and in fluid communication with the proximal coupler 620. In this example, the housing 610 includes a set of slits or the like with flexible and/or deformable portions 615 of the housing 610 disposed therebetween. In some implementations, this arrangement can allow the housing 610 (or at least a portion thereof) to deform or compress when a fluid collection device is inserted into the housing 610. In this manner, a height of the housing 610 is compressed or reduced, which in turn, can decrease a distance between a distal end portion of the housing 610 and the fluid communicator disposed in the housing 610. As such, the fluid communicator can be inserted into a fluid collection device that may not otherwise be inserted into the housing 610 a sufficient distance.

Any of the transfer devices described herein can includes one or more features, portions, and/or arrangements configured to limit and/or prevent undesired access of a fluid communicator. As described above, in some embodiments, a fluid communicator can be a needle with a sharpened distal end that can present a risk of undesirable needle sticks or punctures of a patient and/or user. Thus, any of the transfer devices can include one or more features, portions, and/or arrangements that can enhance and/or increase patient and/or user safety by selectively limiting access to the fluid communicator.

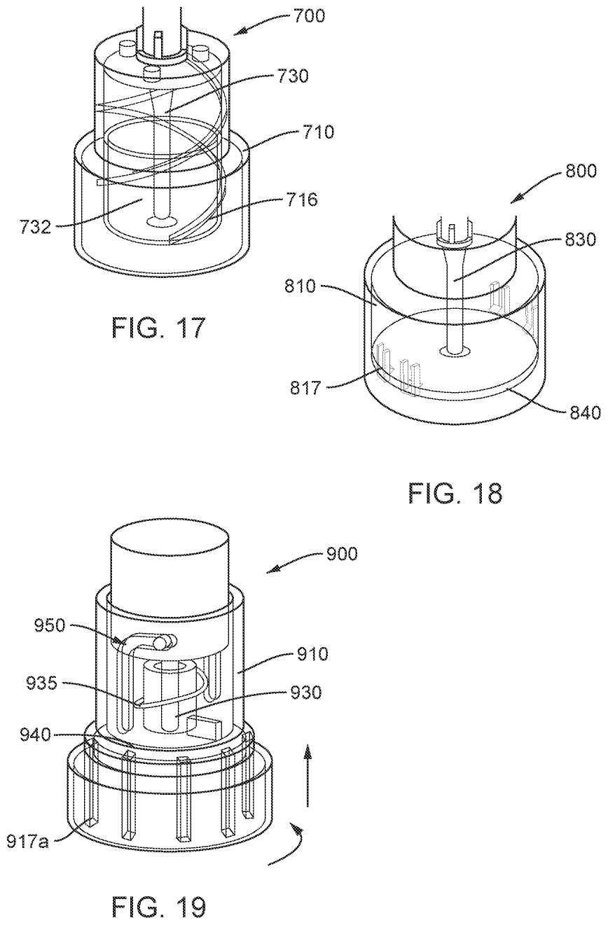

For example, FIG. 17 illustrates a portion of a transfer device 700 according to an embodiment. The transfer device 700 includes a housing 710 and a fluid communicator 730 disposed within the housing 710. In this example, the housing 710 includes and/or defines a spiraled inner track 716 that allows an inner sheath 732 otherwise covering the fluid communicator 730 to twist and compress in response to a fluid collection device being inserted into the housing 710. The twisting and compressing of the inner sheath 732, in turn, exposes a portion of the fluid communicator 730 allowing it to be inserted into the fluid collection device.

FIG. 18 illustrates a portion of a transfer device 800 according to another embodiment. The transfer device 800 includes a housing 810 and a fluid communicator 830 disposed within the housing 810. In this example, the transfer device 800 includes a stage 840 (e.g., a plate, disc, platform, etc.) that selectively limits access to the fluid communicator 830. For example, the stage 840 can selectively engage a set of latches 817 formed by an inner surface of the housing 810 that are configured to at least temporarily maintain the stage 840 in a distal position (shown in FIG. 18). When a fluid collection device is inserted into the housing 810, a surface of the fluid collection device can exert a force on the stage 840 operable to release the stage 840 from the latches 817 and moving the stage 840 in a proximal direction to allow the fluid communicator 830 to be inserted into the fluid collection device. In other implementations, the fluid collection device can engage the latches 817 when inserted into the housing 810. In such implementations, a surface of the fluid collection device can deflect the latches 817 outward to release the stage 840, allowing the stage 840 to move in the proximal direction. In some instances, such an arrangement can be beneficial because a user's finger is unlikely to deflect all the latches 817 (e.g., on two or more sides of the housing 810) at the same time, and thus, is unlikely to release the stage 840.

FIG. 19 illustrates a portion of a transfer device 900 according to another embodiment. The transfer device 900 includes a housing 910 and a fluid communicator 930 disposed within the housing 910. In this example, the transfer device 900 includes a stage 940 that selectively limits access to the fluid communicator 930. The transfer device 900 also includes a bias member 935 (e.g., a spring) that biases and/or at least temporarily maintains the stage in a distal position that limits and/or prevents access to the fluid communicator 930. As shown, the housing 910 can be, for example, a two part configuration including a lock 950 that can transition from a first or locked configuration to a second or unlocked configuration. In addition, an inner surface of the housing 910 can include and/or form one or more engagement or gripping features 917A (e.g., protrusions or ribs formed from a material having a relatively high friction coefficient such as rubber or silicone). In some implementations, a user can, for example, insert a portion of a fluid collection device into the housing 910 such that the engagement or gripping features 917A contact a surface of the fluid collection device. In some instances, after inserting the fluid collection device into the housing 910, a user can rotate the fluid collection device and the friction force between the engagement or gripping features 917A and the surface of the fluid collection device can be sufficient to rotate a first portion of the housing 910 relative to a second portion of the housing 910, thereby transitioning the lock 950 from the first or locked configuration to the second or unlocked configuration. With the lock 950 in the second or unlocked configuration, the first portion of the housing 910 can be allowed to move relative to the second portion of the housing 910, thereby allowing the fluid collection device to be advanced relative to the fluid communicator 930 such that the fluid communicator 930 punctures a surface of the fluid collection device.

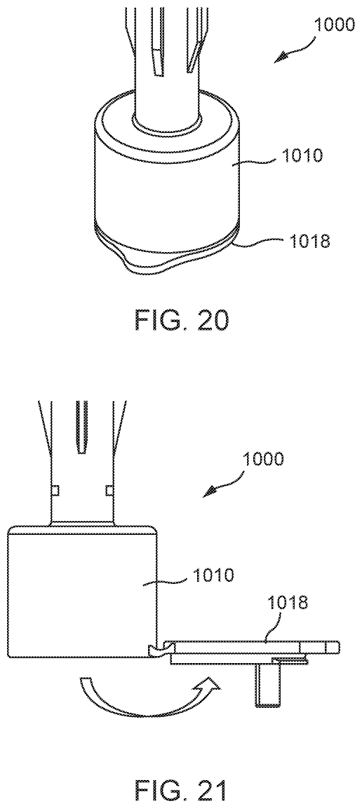

FIGS. 20 and 21 illustrate a portion of a transfer device 1000 according to another embodiment, and shown in a first configuration and a second configuration, respectively. The transfer device 1000 includes a housing 1010 and a fluid communicator (not shown) disposed within the housing 1010. In this example, the transfer device 1000 includes a door 1018 that selectively closes and opens to allow access to the fluid communicator. In some embodiments, for example, the door 1018 can include a tab or catch that can be engaged or grabbed by a user to transition the door 1018 between the closed and open state. As shown in FIG. 20, the transfer device 1000 is in the first configuration when the door 1018 is in a closed state, thereby blocking access to the fluid communicator. As shown in FIG. 21, the transfer device 1000 is in the second configuration when the door 1018 is placed in an open state, thereby allowing access to the fluid communicator. In this embodiment, the door 1018 is shown as including a hinge that allows the door 1018 to swing or rotate between the closed and open states. Moreover, the door 1018 can include a finger guard (e.g., a protrusion, extensions, bump, and/or any other suitable feature) configured to prevent accidental contact with the fluid communicator when opening the door 1018. In other embodiments, a door can be configured to transition between the closed and open states in any suitable manner.

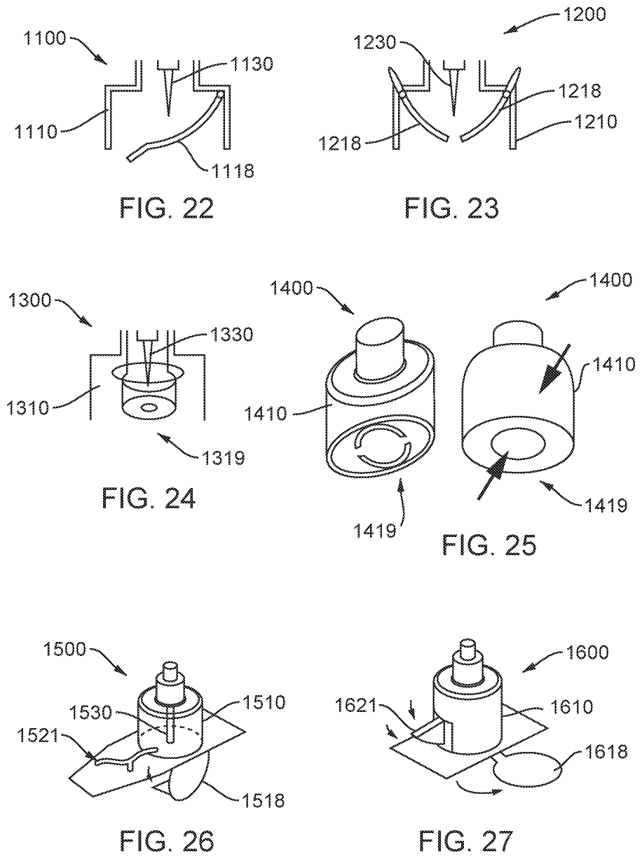

FIG. 22 illustrates a portion of a transfer device 1100 according to another embodiment. The transfer device 1100 includes a housing 1110 and a fluid communicator 1130 disposed within the housing 1110. In this example, the transfer device 1100 includes a door 1118 that selectively closes and opens to allow access to the fluid communicator 1130. Moreover, in this embodiment, the door 1118 can include a catch, tab, protrusion, and/or feature that can be engaged by a portion of a fluid collection device to transfer the door 1118 between the closed and open state, thereby mitigating a risk of contamination associated with a user contacting the door 1118.

FIG. 23 illustrates a portion of a transfer device 1200 according to another embodiment. The transfer device 1200 includes a housing 1210 and a fluid communicator 1230 disposed within the housing 1210. In this example, the transfer device 1200 includes two doors 1218 that collectively transition between a closed and open state to allow access to the fluid communicator 1230. Moreover, in this embodiment, each door 1218 can include an engagement feature disposed outside of the housing 1210 that be manipulated by a user to open or close the doors 1218. For example, in some implementations, a user can exert an inward force on the engagement features, which in turn, move the doors 1218 in an outward direction to an open state.

FIG. 24 illustrates a portion of a transfer device 1300 according to another embodiment. The transfer device 1300 includes a housing 1310 and a fluid communicator 1330 disposed in the housing 1310. In this example, the transfer adapter 1300 includes an inner sheath 1319 that at least partially covers and/or blocks access to the fluid communicator 1330. The housing 1310 can include and/or can form an elliptical opening and/or the like that can selectively receive a portion of the inner sheath 1319. More specifically, at least a portion of the inner sheath 1319 can have a substantially circular shape with a diameter that is greater than a narrow portion of the elliptical opening formed by the housing 1310. In this embodiment, the housing 1310 is configured to be compressed by a user to transform and/or deform a portion of the housing 1310 such that the elliptical opening is squeezed or deformed into a circular opening having a diameter that is greater than a diameter of the inner sheath 1319. In this manner, the user can insert the fluid collection device into the housing 1310 and can compress or move the inner sheath in a proximal direction, and at least partially through the circular opening, to expose a portion of the fluid communicator 1330

FIG. 25 illustrates a portion of a transfer device 1400 according to another embodiment. The transfer device 1400 includes a housing 1410 and a fluid communicator 1430 disposed in the housing 1410. In this example, the housing 1410 has a substantially elliptical shape and/or elliptical opening at a distal end of the housing 1410. Moreover, the distal end of the housing 1410 can form one or more shoulders 1419 that at least partially block or occlude an inner volume of the housing 1410. As described above with reference to the transfer device 1300, in this example, the housing 1410 of the transfer device 1400 is configured to be compressed or squeezed by a user such that the distal end of the housing 1410 deforms to increase a size of an opening formed by the one or more shoulders 1419. For example, the housing 1410 can be compressed or squeezed such that an opening formed by the one or more shoulders 1419 has a shape and/or size that is sufficient to receive at least a portion of a fluid collection device therethrough.

FIG. 26 illustrates a portion of a transfer device 1500 according to another embodiment. The transfer device 1500 includes a housing 1510 and a fluid communicator 1530 disposed in the housing 1510. In this example, the transfer device 1500 includes a door 1518 that is movably or releasably coupled to the housing 1510. As shown, the transfer device 1500 further includes a release mechanism 1521 that can be manipulated by a user to release and/or otherwise allow the door 1518 to transition from a closed state to an open state. For example, the release mechanism 1521 can be a trigger, a latch, an actuator, and/or the like. As described above, the door 1518 can limit and/or block access to the fluid communicator 1530 when the door 1518 is in the closed state and can allow a fluid collection device to access the fluid communicator 1530 when the door 1518 is in the open state.

FIG. 27 illustrates a portion of a transfer device 1600 according to another embodiment. The transfer device 1600 includes a housing 1610 and a fluid communicator 1630 disposed in the housing 1610. In this example, the transfer device 1600 includes a door 1618 that is movably or releasably coupled to the housing 1610. As shown, the transfer device 1600 further includes a release mechanism 1621 that can be manipulated by a user to release and/or otherwise allow the door 1618 to transition from a closed state to an open state. For example, in this embodiment, the release mechanism 1621 can be a cam or the like that can pivot or rotate to release the door 1618. As described above, the door 1618 can limit and/or block access to the fluid communicator 1630 when the door 1618 is in the closed state and can allow a fluid collection device to access the fluid communicator 1630 when the door 1618 is in the open state.

Any of the transfer devices described herein can include one or more features, portions, and/or arrangements configured to enhance, improve, and/or facilitate a user interface. In some implementations, enhancing, improving, facilitating, and/or controlling a user interface can limit and/or mitigate a safety risk and/or risk of contamination by at least partially controlling how a user engages and/or interfaces with at least a portion of the transfer device.

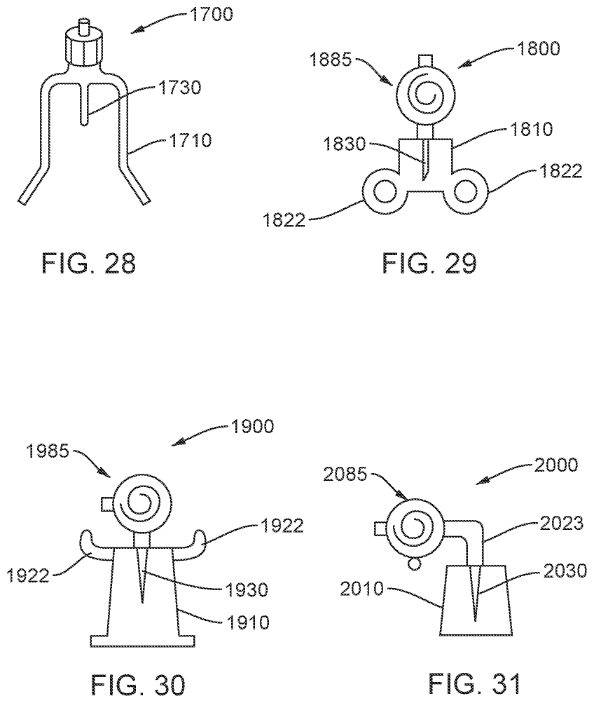

For example, FIG. 28 illustrates a portion of a transfer device 1700 according to another embodiment. The transfer device 1700 includes a housing 1710 and a fluid communicator 1730 disposed in the housing 1710. In this example, the housing 1710 can include an extended and/or flared end portion or flange that can, for example, increase a distance between a tip of the fluid communicator 1730 and a distal edge of the housing 1710. Moreover, in some embodiments, the distal end portion or flange of the housing 1710 can be flared a sufficient amount to allow any suitable fluid collection device to be inserted into the housing 1710 and placed in fluid communicator with the fluid communicator 1730. In this implementations, the housing 1710 and/or the flared distal end portion or flange thereof can improve and/or facilitate a user interface, providing, for example, a horizontal or substantially horizontal (or other surface) that allows a user to exert a downwardly or distally directed force on the housing 1710 facilitating the coupling of the transfer device to a fluid collection device (e.g., sample bottle).

FIG. 29 illustrates a portion of a transfer device 1800 according to another embodiment. The transfer device 1800 includes a housing 1810 and a fluid communicator 1830 disposed in the housing 1810. In this example, a proximal coupler of the transfer device 1800 is physically and fluidically coupled to a diversion and/or sequestration device 1885. A distal end portion of the housing 1810 can include one or more loops 1822 that can be engaged by the fingers of a user. In this manner, the loops 1822 can provide a secure way for the user to engage and/or hold the transfer device 1800, for example, as the user inserts a fluid collection device.

FIG. 30 illustrates a portion of a transfer device 1900 according to another embodiment. The transfer device 1900 includes a housing 1910 and a fluid communicator 1930 disposed in the housing 1910. In this example, a proximal coupler of the transfer device 1900 is physically and fluidically coupled to a diversion and/or sequestration device 1985. A proximal end portion of the housing 1910 can include one or more handles, tabs, hooks, arms, etc. (referred to herein as “handles 1922”) that can be engaged by the fingers of a user. In this manner, the handles 1922 can provide a secure way for the user to engage and/or hold the transfer device 1900, for example, as the user inserts a fluid collection device.

FIG. 31 illustrates a portion of a transfer device 2000 according to another embodiment. The transfer device 2000 includes a housing 2010 and a fluid communicator 2030 disposed in the housing 2010. In this example, a proximal coupler 2023 of the transfer device 2000 is physically and fluidically coupled to a diversion and/or sequestration device 2085. More specifically, in this embodiment, the proximal coupler 2023 forms a bend or the like that can place the diversion and/or sequestration device 2085 in a desired orientation when coupled to the proximal coupler 2023. In some embodiments, for example, the proximal coupler 2023 can form a 90° or substantially 90° bend that can place the diversion and/or sequestration device 2085 in an orthogonal or perpendicular orientation relative to the housing 2010 of the transfer device 2000. In some embodiments, such an arrangement can improve and/or enhance a user interface associated with the transfer device 2000 and/or a visibility of a portion of the diversion and/or sequestration device 2085.

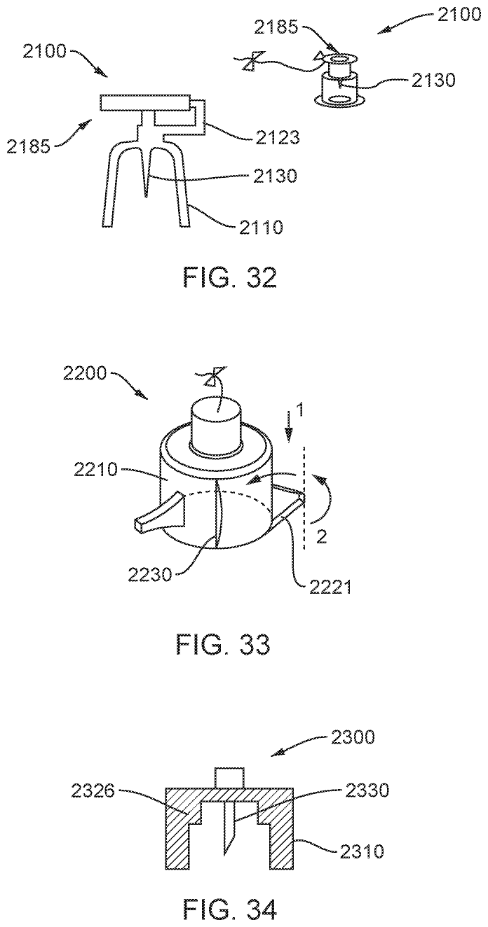

FIG. 32 illustrates a portion of a transfer device 2100 according to another embodiment. The transfer device 2100 includes a housing 2110 and a fluid communicator 2130 disposed in the housing 2110. In this example, a proximal coupler 2123 of the transfer device 2100 is physically and fluidically coupled to a diversion and/or sequestration device 2185. More specifically, in this embodiment, the proximal coupler 2123 forms a bend or the like that can place the diversion and/or sequestration device 2185 in a desired orientation when coupled to the proximal coupler 2123. In some embodiments, for example, the proximal coupler 2123 can form a 90° or substantially 90° bend that can place the diversion and/or sequestration device 2185 in an orthogonal or perpendicular orientation relative to the housing 2110 of the transfer device 2100. Moreover, the orientation and/or arrangement of the diversion and/or sequestration device 2185 relative to the housing 2110 can be such that the fluid communicator 2130 extends from a substantially central portion of the diversion and/or sequestration device 2185. In some embodiments, such an arrangement can improve and/or enhance a user interface associated with the transfer device 2100 and/or a visibility of a portion of the diversion and/or sequestration device 2185.

FIG. 33 illustrates a portion of a transfer device 2200 according to another embodiment. The transfer device 2200 includes a housing 2210 and a fluid communicator 2230 disposed in the housing 2210. In this example, a proximal coupler 2223 of the transfer device 2200 can be physically and fluidically coupled to a diversion and/or sequestration device (not shown). A distal end portion of the housing 2210 can include one or more handles, tabs, hooks, arms, etc. (referred to herein as “handles 2221”) that can be engaged by the fingers of a user. In this manner, the handles 2221 can provide a secure way for the user to engage and/or hold the transfer device 2200, for example, as the user inserts a fluid collection device. Moreover, one or more of the handles 2221 can be reconfigurable between a first state or configuration and a second state or configuration. For example, in some implementations, at least one of the handles 2221 can have a first state and/or configuration in which the handle 2221 extends from a side of the housing 2210 and a second state and/or configuration in which the handle 2221 can be compressed or reconfigured and inserted into a portion of the housing 2210 to block and/or substantially limit access to the fluid communicator 2230. In this manner, the handles 2221 can be configured to enhance a user interface associated with the transfer device 2200 as well as provide additional safety features that protect against undesirable contact with the fluid communicator 2230.

FIG. 34 illustrates a portion of a transfer device 2300 according to another embodiment. The transfer device 2300 includes a housing 2310 and a fluid communicator 2330 disposed in the housing 2310. In this example, the housing 2310 can have an inner surface (or a portion thereof) that includes an overmolded section 2326 formed from a relatively soft and/or relatively high friction material. In this manner, the overmolded section 2326 can contact a surface of the fluid collection device when the fluid collection device is inserted into the housing 2310 and a friction force therebetween can be sufficient to at least temporarily retain the fluid collection device in a fixed position relative to the transfer device 2310. In some instances, such an arrangement, for example, can allow a user to release his or her grip on the transfer device 2310 without the fluid collection device decoupling from or falling out of the transfer device 2310.

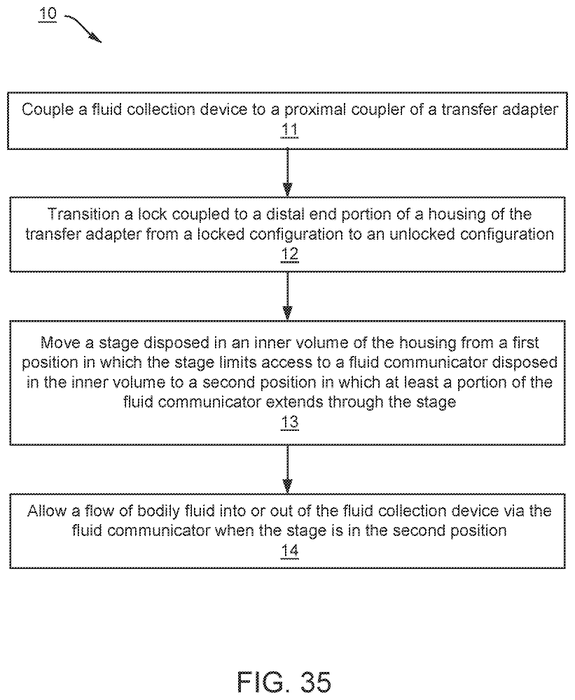

FIG. 35 illustrates a flowchart of a method 10 for using a transfer adapter according to an implementation. The transfer adapter can be substantially similar to any of the transfer adapters described herein. In some implementations, the transfer adapter can be substantially similar to and/or can be used in a substantially similar manner as the transfer adapter 200 (e.g., can include and/or can be used with, for example, an optional distal coupler and/or the like). In some implementations, the transfer adapter can be substantially similar to and/or can be used in a substantially similar manner as the transfer adapter 300 (e.g., does not include and/or is not used with, for example, the optional distal coupler and/or the like). In either implementation, the transfer adapter can include at least a housing, a fluid communicator disposed in the housing, a lock coupled to a distal end portion of the housing, and a stage movable within the housing.

As shown, the method 10 includes coupling a fluid collection device to a proximal coupler of the transfer adapter, at 11. The fluid collection device can be any of those described herein. For example, in some implementations, the fluid collection device can be a syringe, as described above with reference to the transfer adapter 200 shown in FIGS. 2-10. In other implementations, the proximal coupler can be coupled, directly or indirectly, to a bodily fluid source (e.g., via a needle, catheter, access device, transfer device, diversion device, sequestration device, and/or any other suitable device). For example, in some implementations, the proximal coupler can be coupled to a fluid transfer device such as any of those described in the '420 Patent, the '241 Patent, the '950 Patent, the '774 Patent, the '576 Patent, the '864 Patent, the '240 Publication, the '117 Publication, the '074 Publication, the '087 Publication, the '303 Publication, the '039 Publication, and/or the '732 Application.

The lock coupled to the distal end portion of the housing is transitioned from a locked configuration to an unlocked configuration, at 12. As described above with reference to the adapters 100, 200, and/or 300, the lock can be rotated relative to the housing to transition between the locked configuration and the unlocked configuration. In other implementations, the lock can be moved in a linear motion, can be a push button or toggle, and/or can be transitioned in any other manner. As described above, the lock in the locked configuration can selectively engage the stage to maintain the stage in a distal or biased position in which the stage limits and/or substantially prevents access to the fluid communicator via the distal end portion of the housing (e.g., the stage can include a seal or the like that can seal off the open distal end portion of the housing from the fluid communicator disposed in the housing. In some implementations, such a seal, block, and/or isolation can be a collective result of, for example, the stage and a sheath that can at least temporarily surround a distal end portion of the fluid communicator.

Transitioning the lock from the locked configuration to the unlocked configuration can disengage the lock from the stage, thereby allowing the stage to be moved in response to an applied force. The method 10 includes moving the stage from a first or distal position in which the stage limits access to the fluid communicator disposed in the inner volume of the housing to a second or proximal position in which at least a portion of the fluid communicator extends through, beyond, and/or distal to the stage, at 13. As such, when the stage is in the second position, a flow of bodily fluid is allowed into or out of the fluid collection device via the fluid communicator, at 14.

In some implementations, for example, the stage can be moved in response to a distal coupler being coupled to the distal end portion of the housing, as described above with reference to the transfer adapter 200. In such implementations, the proximal coupler can be coupled to a syringe or the like and the distal coupler can be coupled (directly or indirectly) to a bodily fluid source. Thus, a user can manipulate the syringe to draw bodily fluid from the bodily fluid source, into and through the distal coupler, through the fluid communicator and proximal coupler, and into the syringe.

In other implementations, the stage can be moved in response to a second fluid collection device being coupled to and/or inserted into the distal end portion of the housing, as described above with reference to the transfer adapter 300. In such implementations, the second fluid collection device can be, for example, a culture bottle or the like and the proximal coupler can be coupled (directly or indirectly) to a bodily fluid source. Thus, a user can draw bodily fluid from the bodily fluid source, into and through the proximal coupler, through the fluid communicator, and into the culture bottle.

While the method 10 is described above as allowing the flow of bodily fluid from a bodily fluid source and into a syringe and/or second collection device (e.g., culture bottle), in some implementations, the method 10 can also be performed by and/or otherwise can include transferring bodily fluid from a syringe into a second collection device. For example, a volume of bodily fluid can be drawn from a bodily fluid source into the syringe using the transfer adapter with the optional distal coupler, as described above. After receiving a desired volume of bodily fluid, the optional distal coupler can be removed from the transfer adapter and the stage can be allowed to return to the distal, biased, or first position. In some instances, the user optionally can transition the lock to the locked configuration.

In this implementations, it is desirable to transfer at least a portion of the bodily fluid disposed in the syringe into a separate fluid collection device such as a sample bottle, culture bottle, testing apparatus, and/or the like. Thus, if not already in the second or unlocked configuration, the user can transition the lock back to the second or unlocked configuration and can insert a portion of a culture bottle into the distal end portion of the housing, as described above with reference to the use of the transfer adapter without the optional distal coupler. Inserting the culture bottle or the like results in a surface thereof being placed in contact with the stage and, with the lock in the second or unlocked configuration, further insertion moves and/or transition the stage from the distal position toward the proximal position. As such, an unsheathed distal end portion of the fluid communicator can extend distally relatively to the stage and can puncture and/or otherwise be inserted into a portion of the culture bottle, thereby establishing fluid communication between the syringe and the culture bottle. Thus, the user can manipulate the plunger of the syringe or rely on a vacuum charge (e.g., negative pressure differential) of the culture bottle to transfer a desired volume of bodily fluid from the syringe to the culture bottle via the transfer adapter without a need for additional devices and/or components that may otherwise introduce points of potential contamination.

While various embodiments have been described above, it should be understood that they have been presented by way of example only, and not limitation. Where schematics and/or embodiments described above indicate certain components arranged in certain orientations or positions, the arrangement of components may be modified. While the embodiments have been particularly shown and described, it will be understood that various changes in form and details may be made. Although various embodiments have been described as having particular features, concepts, and/or combinations of components, other embodiments are possible having any combination or sub-combination of any features, concepts, and/or components from any of the embodiments described herein.

The specific configurations of the various components can also be varied. For example, the size and specific shape of the various components can be different from the embodiments shown, while still providing the functions as described herein. In some embodiments, varying the size and/or shape of such components may reduce an overall size of the device and/or may increase the ergonomics of the device without changing the function of the device. In some embodiments, the size and/or shape of the various components can be specifically selected for a desired or intended usage. Thus, it should be understood that the size, shape, and/or arrangement of the embodiments and/or components thereof can be adapted for a given use unless the context explicitly states otherwise.

For example, while the stages 240 and 340 are described above as being moved or transitioned by the bias members 235 and 335, respectively, in other embodiments, the stages 240 and/or 340 can be manually actuated, moved, and/or transitioned. For example, in some embodiments, a transfer adapter can include an actuator, tab, slider, button, and/or other suitable feature that is directly or indirectly coupled to a stage. In such embodiments, a user can exert a force on the feature to move the feature, which in turn, can move the stage between a first configuration and/or position (e.g., a distal or locked position) and a second configuration and/or position (e.g., a proximal or unlocked position).

While the locks 150, 250, and/or 350 are described above as being rings that are coupled to the housings 110, 210, and/or 310, respectively, and configured to be rotated relative thereto, in other embodiments, a transfer adapter or device can include any suitable lock. For example, in some embodiments, a lock can be configured to move in a translational motion between a locked and unlocked configuration. In other embodiments, a lock can be configured to transition between any number of states substantially without moving (e.g., without translating or rotating). In still other embodiments, a transfer device need not include a lock. In such embodiments, for example, a stage can be manually actuated and/or at least temporarily maintained in a fixed position based on an amount of friction between one or more components, and/or via any other suitable means. Similarly, in some embodiments, a distal coupler or any other suitable connection (e.g., the distal coupler 225) can be at least temporarily coupled to a housing via a friction force or any other suitable coupling or engagement. In some embodiments, a transfer device need not include a lock or a stage. In such embodiments, any of the features, concepts, and/or embodiments (or portions thereof) can be used to limit and/or block access to a fluid communicator to mitigate a potential source of contamination.

While the proximal coupler 220 of the transfer device 200 is described above as being coupled to the connector 292 of the syringe 290, in other embodiments, a transfer adapter may be coupled to any suitable portion of a syringe and/or other device. For example, in some embodiments, a syringe can be configured such that a plunger, actuator, and/or the like is in fluid communication with an inner volume or fluid reservoir of the syringe. In such embodiments, the plunger, actuator, and/or the like can include a port or connector that can be coupled to a coupler of a transfer device or adapter similar to the proximal couplers 120, 220, and/or 320 described herein. More specifically, while the transfer adapter 300 is described above as connecting to a fluid transfer device via the proximal coupler 320, in some implementations, the proximal coupler 320 can be coupled to such a port of a syringe. In this manner, the transfer adapter 320 can be coupled to an actuator or plunger of the syringe and can extend from and/or otherwise can be disposed on a proximal side of the syringe. In some embodiments, such an arrangement can be substantially similar to a syringe and transfer adapter combination described, for example, in U.S. Patent Publication No. 2016/0361006 (“the '006 Publication”) entitled, “Devices and Methods for Syringe-Based Fluid Transfer for Bodily-Fluid Sampling,” filed Jun. 13, 2016, the disclosure of which is incorporated herein by reference in its entirety.

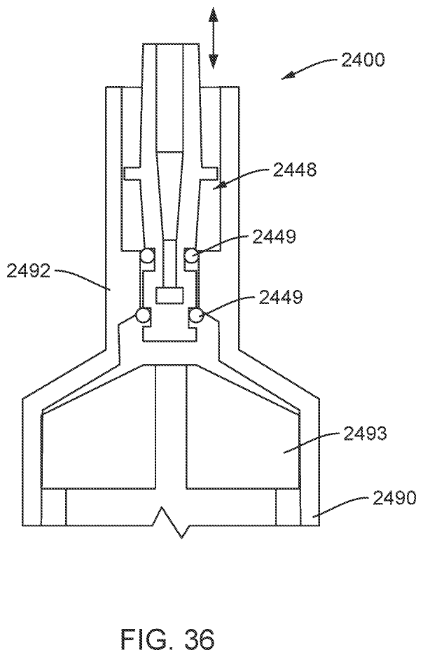

In some implementations, a syringe can include a valve or other flow control device that can control, modulate, regulate, enable/disable, etc. flow into and/or through the syringe which can facilitate the use of a transfer adapter coupled to a proximal side of the syringe (described above). In some implementations, a valve or the like can be integrated into or as a separate component coupled to or included in, for example, a coupler or connector of a syringe or other fluid collection device. For example, FIG. 36 illustrates a portion of a syringe 2490 that includes a coupler 2492 including a valve 2448. In this embodiment, the valve 2448 can be movably disposed in the coupler 2492 of the syringe 2490. The valve 2448, for example, can include a pair of seals 2449 that are spaced apart by a predetermined distance to selectively engage portions of an inner surface of the syringe 2490 (or coupler 2492 thereof). The valve 2448 can define a channel with an outlet disposed between the seals 2449.

In this embodiment, prior to the coupler 2492 of the syringe 2490 being coupled to a corresponding coupler of another device, the valve 2448 can be in a first configuration and/or position (e.g., a distal position), in which the outlet of the channel is disposed within an annular space defined by the inner surface of the syringe 2490, an outer surface of the valve 2448, and the two seals 2449. Furthermore, coupling the coupler 2492 to a corresponding coupler of another device can be operable in transitioning and/or moving the valve 2448 from the first configuration and/or position (e.g., the distal position) to a second configuration and/or position (e.g., a proximal position).

In some implementations, the proximal movement of the valve 2448 can result in the proximal seal 2449 being moved away from the inner surface of the syringe 2490, thereby disengaging. As such, the channel of the valve 2448 is now in fluid communication with an inner volume of the syringe 2490 via the outlet. Thus, a user can manipulate the syringe 2490 by moving an actuator or plunger of the syringe 2490 in a proximal direction, which produces a negative pressure differential or suction force within the syringe 2490 operable to draw a flow of bodily fluid through the channel and the outlet of the valve 2448 and into the inner volume of the syringe 2490.

As described above, the actuator and/or plunger 2493 of the syringe 2490 can define a channel, lumen, etc., configured to allow sampling of the volume of bodily fluid contained in the syringe 2490, as described, for example, in the '006 Publication. For example, after transferring a volume of bodily fluid into the syringe 2490 (e.g., by moving the plunger 2493 in a proximal direction away from the valve 2448), a transfer adapter (e.g., the transfer adapters 100, 200, and/or 300) can be coupled to the actuator and/or plunger 2493 such that a fluid communicator of the transfer adapter is in fluid communication with the channel extending through the plunger 2493. Moreover, a fluid collection device such as, for example, a culture bottle or the like can be inserted into the transfer adapter such that the fluid communicator is in fluid communication with an inner volume of the culture bottle. In this manner, the plunger 2493 can be moved, for example, in a distal direction, thereby increasing a pressure within the syringe 2490 that is operable in expelling at least a portion of the bodily fluid contained therein into and through the channel of the plunger 2493, into and through the fluid communicator, and into the culture bottle. The valve 2448 can facilitate such sampling because the increase in the pressure within the syringe 2490 can move the valve 2448 into a distal position (if not already in the distal position) that is operable in fluidically isolating and/or sealing the opening of the valve 2448 from the inner volume of the syringe 2490 proximal to at least one seal of the valve 2448. Thus, the valve 2448 prevents bodily fluid within the syringe 2490 from being expelled through the coupler 2492 of the syringe, thereby facilitating and/or allowing the sampling from the syringe 2490 (e.g., via the transfer adapter and fluid collection device coupled thereto).

Any number of portions and/or features of the embodiments described herein can be used (or modified for use) with any suitable fluid transfer devices, fluid collection devices, fluid storage devices, and/or the like. For example, in some implementations, the proximal adapter 320 of the transfer device 300 may be physically and/or fluidically coupled to a syringe, as described above with reference to the transfer device 200. Alternatively, the proximal adapter 320 can be physically and/or fluidically coupled to any other suitable device. For example, in some implementations, a transfer device or adapter can be coupled to a device configured to collect, divert, sequester, isolate, etc. an initial volume of bodily fluid, which may be more likely to contain contaminant dislodged during venipuncture or the like. In some instances, contaminants such as dermally residing microbes or the like can be included in the sequestered initial amount of bodily fluid such that subsequent amount(s) of bodily fluid transferred to and/or through the transfer device or adapter are substantially free from contaminants associated with accessing the bodily fluid source (e.g., a vein). Examples of such devices can include, for example, any of the devices and/or embodiments described in the '420 Patent, the '241 Patent, the '950 Patent, the '774 Patent, the '576 Patent, the '864 Patent, the '240 Publication, the '117 Publication, the '074 Publication, the '087 Publication, the '303 Publication, the '039 Publication, and/or the '732 Application, the disclosures of which are incorporated herein by reference in their entireties.

While one or more methods or method steps of using the devices may be described herein as including certain ordered steps, in other embodiments, the ordering of certain events and/or procedures in any of the methods or processes described herein may be modified and such modifications are in accordance with the variations of the invention. Additionally, certain events and/or procedures may be performed concurrently in a parallel process when possible, as well as performed sequentially as described above. Certain steps may be partially completed or may be omitted before proceeding to subsequent steps. For example, while the devices are described herein as transitioning from a first state or configuration to a second state or configuration in a discrete operation or the like, it should be understood that the devices described herein can be configured to automatically and/or passively transition from the first state or configuration to the second state or configuration and that such a transitioning may occur over a period of time. In other words, the transitioning from the first state to the second state may, in some instances, be relatively gradual.

What is claimed:

1. An apparatus, comprising:
a housing having a proximal end portion and a distal end portion and defining an inner volume;
a distal coupler being coupleable to the distal end portion of the housing, the distal coupler configured to be placed in fluid communication with a bodily fluid source;
a fluid communicator disposed in the inner volume of the housing; and
a lock coupled to the housing, the lock having a first configuration in which the lock couples the distal coupler to the housing such that a portion of the fluid communicator extends through a seal of the distal coupler to establish fluid communication between the distal coupler and the proximal end portion of the housing, the lock configured to be transitioned from the first configuration to a second configuration to allow the distal coupler to be removed from the housing and with the distal coupler removed, the lock configured to be transitioned from the second configuration to the first configuration to limit access to the fluid communicator via the distal end portion of the housing.

2. The apparatus of claim 1, wherein the lock includes a stage that is in at least temporarily maintained in a first position when the lock is in the first configuration and allowed to be moved to a second position when the lock is in the second configuration, the stage limiting access to the fluid communicator when in the first position.

3. The apparatus of claim 2, wherein the lock is configured to be transitioned from the second configuration to the first configuration after removing the distal coupler from the housing to prevent the stage from being moved from the first position toward the second position.

4. The apparatus of claim 3, wherein the fluid communicator is a needle having a sharpened distal end portion, the stage configured to limit access to the sharpened distal end portion of the needle when in the first position.

5. The apparatus of claim 1, further comprising:
a proximal coupler coupled to the proximal end portion of the housing and fluidically coupled to the fluid communicator, the proximal coupler configured to couple to a fluid collection device, the fluid collection device configured to receive a volume of bodily fluid via the fluid communicator when the lock is in the first configuration.

6. The apparatus of claim 5, wherein the distal end portion of the housing is configured to receive a portion of a sample reservoir when the lock is in the second configuration and the distal coupler is removed from the housing, the fluid communicator configured to extend into the sample reservoir when the portion of the sample reservoir is disposed in the housing to allow at least a portion of the volume of bodily fluid to be transferred from the fluid collection device to the sample reservoir via the fluid communicator.

7. An apparatus, comprising:
a housing having a proximal end portion and a distal end portion and defining an inner volume, the proximal end portion having a proximal coupler;
a fluid communicator disposed in the inner volume of the housing and fluidically coupled to the proximal coupler;
a stage disposed in the housing and movable between a first position and a second position; and
a bias member disposed in the housing and in contact with a proximal side of the stage, the bias member configured to bias the stage in the first position such that the stage substantially prevents access to the fluid communicator via the distal end portion of the housing, the bias member allowing the stage to be moved to the second position in response to a force exerted on a distal side of the stage such that a portion of the fluid communicator extends through the stage, thereby allowing access to the fluid communicator via the distal end portion of the housing.

8. The apparatus of claim 7, wherein the fluid communicator is a needle having a sharpened distal end portion, the stage configured to limit access to the sharpened distal end portion of the needle when in the second position.

9. The apparatus of claim 7, wherein the proximal coupler is configured to couple to a fluid collection device, the apparatus further comprising:
a lock coupled to the distal end portion of the housing and transitionable between a locked configuration and an unlocked configuration, the fluid communicator allowed to transfer bodily fluid to the fluid collection device when the lock is in the unlocked configuration and the proximal coupler is coupled to the fluid collection device.

10. The apparatus of claim 9, wherein the fluid collection device is a syringe.

11. The apparatus of claim 7, further comprising:
a distal coupler removably coupleable to the distal end portion of the housing, the distal coupler is operable in placing the stage in the second position when the distal coupler is coupled to the distal end portion of the housing.

12. The apparatus of claim 11, wherein the lock couples the distal coupler to the distal end portion of the housing when in the locked configuration, and
the lock allows the distal coupler to be removed from the distal end portion of the housing when in the unlocked configuration.

13. The apparatus of claim 11, wherein the distal coupler includes a seal, at least a portion of the fluid communicator extends through the seal of the distal coupler when the distal coupler is coupled to the distal end portion of the housing to fluidically couple the distal coupler to the fluid communicator.

14. A method of using a transfer adapter, comprising:
coupling a fluid collection device to a proximal coupler of the transfer adapter, the transfer adapter having a housing with a proximal end portion and a distal end portion, the proximal coupler disposed along the proximal end portion, the transfer adapter including a fluid communicator disposed in an inner volume of the housing and fluidically coupled to the proximal coupler;
transitioning a lock coupled to the distal end portion of the housing from a locked configuration to an unlocked configuration;
moving a stage disposed in the inner volume of the housing from a first position in which the stage limits access to the fluid communicator via the distal end portion of the housing to a second position in which at least a portion of the fluid communicator extends through the stage; and
allowing a flow of bodily fluid into or out of the fluid collection device coupled to the proximal coupler via the fluid communicator when the stage is in the second position.

15. The method of claim 14, further comprising:
inserting a portion of a sample reservoir into the distal end portion of the housing while the lock is in the unlocked configuration, the moving of the stage being in response to the inserting of the portion of the sample reservoir.

16. The method of claim 15, wherein the fluid collection device is a needle in fluid communication with a bodily fluid source, the allowing the flow of bodily fluid into or out of the fluid collection device via the fluid communicator includes allowing a flow of bodily fluid from the needle to the sample reservoir through the fluid communicator.

17. The method of claim 14, wherein the fluid collection device is a syringe.

18. The method of claim 17, further comprising:
inserting a portion of a distal coupler into the distal end portion of the housing while the lock is in the unlocked configuration, the moving of the stage being in response to the inserting of the portion of the distal coupler; and
transitioning the lock from the unlocked configuration to the locked configuration to temporarily couple the distal coupler to the housing.

19. The method of claim 18, wherein the inserting of the distal coupler is such that at least a portion of the fluid communicator extends through a seal in the distal coupler to fluidically couple the distal coupler to the fluid communicator.

20. The method of claim 19, wherein the distal coupler is in fluid communication with a bodily fluid source, the allowing of the flow of bodily fluid into or out of the fluid collection device via the fluid communicator includes allowing a flow of bodily fluid from the bodily fluid source to the syringe through the fluid communicator.
